(19) United States
(12) Patent Application Publication
(10) Pub. No.: US 2026/0240852 A1
(43) Pub. Date: Aug. 20, 2026

(54) Use of Oral JAK Kinase Inhibitors to Treat Lichen Planus

(71) Applicant: Mayo Foundation for Medical Education and Research, Rochester, MN (US)

(72) Inventor: Aaron R. Mangold, Scottsdale, AZ (US)

(21) Appl. No.: 19/164,861

(22) PCT Filed: Mar. 15, 2024
(86) PCT No.: PCT/US2024/020149

§ 371 Date: Sep. 12, 2025

Related U.S. Application Data
(60) Provisional application No. 63/453,065, filed on Mar. 17, 2023.

(51) Int. Cl. A61K 31/519

(52) U.S. Cl. CPC A61K 31/519

(57) ABSTRACT

Provided herein are methods for treating cutaneous lichen planus in a mammal in need thereof, said method comprising orally administering, to said mammal, a composition comprising a janus kinase (JAK) inhibitor, wherein said administering results in a reduction in total lesion count from baseline.

### TECHNICAL FIELD

This document relates to methods and materials for oral treatment of lichen planus (LP) using orally administered janus kinase (JAK) inhibitors. For example, this document relates to methods and materials for oral treatment of LP using orally administered baricitinib.

### STATEMENT OF FEDERALLY FUNDED RESEARCH

None.

### INCORPORATION-BY-REFERENCE OF MATERIALS FILED ON COMPACT DISC

The Sequence Listing in an XML file, named as ______.xml of ______ KB, created on ______, and submitted to the United States Patent and Trademark Office via Patent Center, is incorporated herein by reference.

### BACKGROUND

LP is an inflammatory skin disease characterized by purple, polygonal, pruritic, papules and plaques on the skin, inflammation scaling and hair loss on the scalp, and ulcerative and reticulated plaques and patches on mucosal surfaces. The worldwide prevalence of LP is about 1%. The disease results in well-marginated, flat-topped, red-violet polygonal lesions that can be distributed in groups and can affect the flexural aspects of the arms and legs, the scalp, and mucosal surfaces. Currently available treatments include corticosteroids (topical, IL, oral), topical calcineurin inhibitors, phototherapy, sulfasalazine, acitretin, methotrexate, but no disease-specific medications for LP exist.

### SUMMARY

This document provides, inter alia, methods of treating mammals (e.g., humans) having LP with an orally administered JAK inhibitor such as, for example, baricitinib.

In one aspect, this document features a method for treating cutaneous LP in a mammal in need thereof. The method can include or consist essentially of orally administering, to the mammal, a composition comprising a Janus Kinase (JAK) inhibitor, where the administering results in a reduction in total lesion count from baseline. The mammal can be a human (e.g., an adult human). The JAK inhibitor can be baricitinib, ruxolitinib, tofacitinib, upadacitinib, fedratinib, pacritinib, momelotinib, abrocitinib, delgocitinib, filgotinib, peficitinib, povorcitinib, deucravacitinib, brepocitinib, SHR0302, or itacitinib. The administering can result in an improvement in the mammal's Index Treatment and Control Lesion by modified Clinical Assessment Scale of Severity for Index Lesion Signs and Symptoms (CAILS) score from baseline. The administering can result in a reduction in the mammal's total lesion count from baseline. The administering can result in a reduction in the mammal's percentage of the Body Surface Area of the cutaneous lichen planus involvement as compared to baseline. The administering can result in a Physician Global Cutaneous Lichen Planus Assessment of 0 (clear), 1 (almost clear), or 2 (significant improvement). The administering can result in a reduction in the mammal's pruritus Numerical Rating Scale score as compared to baseline. The mammal can have clinical and histological features of LP. The mammal can have a Body Surface Area of lichen planus involvement of up to 20% at baseline. The mammal can have a Body Surface Area of lichen planus involvement of 2% to 20% at baseline. The mammal can have a minimum of 4 lesions of lichen planus at baseline. The mammal can have a minimum of 10 lesions of lichen planus at baseline. The mammal can have had lichen planus for at least 8 months. The mammal can have failed to achieve an adequate response to prior therapy for lichen planus. The prior therapy can be selected from the group consisting of one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide; topical tacrolimus and pimecrolimus; systemic immunosuppressant; oral metronidazole; oral sulfasalazine; and oral retinoid. In another aspect, the baricitinib, ruxolitinib, tofacitinib, upadacitinib, fedratinib, pacritinib, momelotinib, abrocitinib, delgocitinib, filgotinib, peficitinib, povorcitinib, deucravacitinib, brepocitinib, SHR0302, or itacitinib is deuterated.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more cases of the invention are set forth in the accompanying drawings and the description below. Other features, objects, and advantages of the invention will be apparent from the description and drawings, and from the claims.

### DESCRIPTION OF DRAWINGS

For a more complete understanding of the features and advantages of the present invention, reference is now made to the detailed description of the invention along with the accompanying figures and in which:

FIG. 1 is a schematic illustrating LP pathogenesis.

FIG. 2A is a table indicating the results of a trial of orally administered baricitinib in subjects having LP, assessed by Physician Global Assessment (PGA). FIG. 2B is a graph plotting the results shown in FIG. 2A.

FIG. 3 is a table indicating the secondary endpoint results of the trial.

FIG. 4 includes images showing LP lesions on the skin of a study participant at baseline (week 0), and at weeks 12 and 16.

FIG. 5 shows image of cutaneous LP response to baricitinib (Day 0 vs. Week 16).

FIGS. 6A to 6D shows that LP is an IFN driven disease process. (FIG. 6A) Volcano plot of bulkRNA-seq data comparing lesional vs. nonlesional LP skin at Day 0 (n=10 and 9, respectively) (red color shows FDR<0.05 and Log 2 FC>1 or <−1, blue is FC<1 and >−1 and FDR<0.05), green is FC>1 or <−1 and FDR>0.05). (FIG. 6B) Enriched GO categories in DEGs between lesional vs. nonlesional LP skin (red, and blue colors represent enriched GO categories amongst increased vs. decreased DEGs, respectively, p<0.05). (FIG. 6C). Cellular deconvolution of fibroblasts, keratinocytes, myeloid cells, and T cells on the Visium 10× spatial expression platform (representative of n=9). (FIG. 6D) IHC of the T cell marker CD3, pSTAT2, IFN-γ, and CXCL9 (representative of n=9, scale bar=100 μm).

FIGS. 7A to 7G shows the cellular composition of LP and effect of baricitinib treatment. (FIG. 7A) Changes in gene expression in interferon signature genes at baseline and week 2. (FIG. 7B) Cell proportions at baseline and week 2. (FIG. 7C) Single-cell data from baseline (Day 0) and at week 2 in the patient cohort (n=10, 10). (FIG. 7D) Single-cell data from the LP cohort defines 6 distinct keratinocyte clusters, including two basal cell states. (FIG. 7E) The proportion of each keratinocyte subset at baseline and week 2 of treatment. (FIG. 7F) Enriched GO categories in the two basal keratinocyte clusters. (FIG. 7G) expression of MHC class I and class II molecules in the different keratinocyte compartments at different time points.

FIG. 8A to 8F show T cell function in LP. (FIG. 8A) Six T cell subsets are found in LP skin. (FIG. 8B) The proportion of T cell subsets at baseline and week 2 of treatment. (FIG. 8C) IFNG expression in T cells subsets in LP skin. (FIG. 8D) Type II IFN signaling network in LP skin. (FIG. 8E) Oligoclonality of CXCL13+ CD8+ T cells in LP skin (3 representative patients). (FIG. 8F) Immunofluorescence of CD3 (green) and CXCL13 (red) in LP skin, showing co-localization of double-positive CXCL13+-CD8+ T cells adjacent to the epidermal-dermal junction (white broken line) (representative image of n=3).

FIGS. 9A to 9C show regulatory T cells in a new pathogenic T-cell identified in LP. FIG. 9A shows Clean T-regulatory population by single cell sequencing. FIG. 9B shows the co-localization of CXCL13 and T-regulatory cells. FIG. 9C shows a similar change of CXCL13 and T-regulatory cells with treatment.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

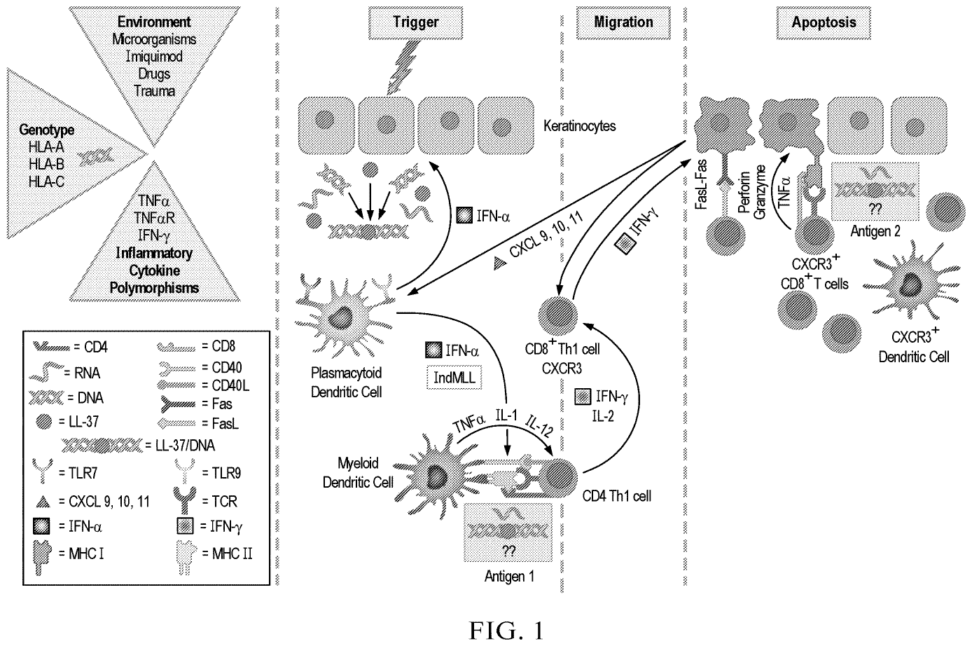

LP pathogenesis is illustrated in FIG. 1. LP is an idiopathic T-cell-mediated process without a clear auto-antigen. Interferon gamma (IFNγ) is a key mediator in disease pathogenesis, attracting lymphocytes and plasmacytoid dendritic cells to the epidermis, and stimulating lymphocyte and keratinocyte interaction, which leads to keratinocyte death since IFNγ primed keratinocytes are vulnerable to CD8⁺ T-cell cytotoxicity. Stimulated CD4⁺ T-cells release IFNγ and in turn, the cytokine stimulates CD8⁺ T-cells, which essentially creates a loop that enhances keratinocyte apoptosis.

This document provides methods for treating cutaneous LP in a mammal (e.g., a human) in need thereof. The methods can include administering to the mammal an oral formulation containing a JAK (e.g., JAK1 and/or JAK2) inhibitor. Any suitable JAK inhibitor can be used in the methods provided herein. JAK inhibitors that can be sued in the methods provided herein include, without limitation, baricitinib (INCB028050; Selleckchem), ruxolitinib (Jakafi; Incyte), tofacitinib (Xeljanz; Pfizer), upadacitinib (Rinvoq; AbbVie), fedratinib (Inrebic; Bristol Myers Squibb), pacritinib (Vonjo; CTI), momelotinib (GSK), abrocitinib (Cibinqo; Pfizer), delgocitinib (LEO Pharma), filgotinib (Jyseleca; Galapagos NV), peficitinib (Smyraf; Astellas Pharma), deucravacitinib (Sotyktu; Bristol Myers Squibb), brepocitinib (Priovant Therapeutics, Inc.), SHR0302 (Reistone Biopharma), and itacitinib (Incyte), and povorcitinib (Incyte). In some cases, an oral formulation containing a JAK inhibitor can be administered to a mammal having LP one or more (e.g., two, three, or more than three) times per day. In some cases, the baricitinib (INCB028050; Selleckchem), ruxolitinib (Jakafi; Incyte), tofacitinib (Xeljanz; Pfizer), upadacitinib (Rinvoq; AbbVie), fedratinib (Inrebic; Bristol Myers Squibb), pacritinib (Vonjo; CTI), momelotinib (GSK), abrocitinib (Cibingo; Pfizer), delgocitinib (LEO Pharma), filgotinib (Jyseleca; Galapagos NV), peficitinib (Smyraf; Astellas Pharma), deucravacitinib (Sotyktu; Bristol Myers Squibb), brepocitinib (Priovant Therapeutics, Inc.), SHR0302 (Reistone Biopharma), and itacitinib (Incyte), or povorcitinib (Incyte) can be deuterated.

Baricitinib is a compound that can be used to treat rheumatoid arthritis, alopecia areata, and COVID-19, and also can be used to treat LP as described herein. Baricitinib is a selective inhibitor that blocks JAK subtypes JAK1 and JAK2 (Fridman et al., J Immunol, 184(9):5298-5307, 2010). Baricitinib can modulate the JAK-STAT pathway at the point of JAK1 and JAK2 by preventing phosphorylation of STATs, thus reducing the subsequent production of IFNγ. By targeting the JAK-STAT pathway with baricitinib, the downstream transcriptional changes that lead to Th1-inflammatory response, cytotoxic activity of CD8⁺ T-cells, and keratinocyte destruction by this T-cell subset.

In some cases, the mammal can achieve a reduction in total lesion count from baseline. In some cases, the reduction is statistically significant. In some cases, the mammal can achieve a statistically significant reduction in total lesion count from baseline 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks from the onset of treatment with a JAK inhibitor (e.g., baricitinib).

In some cases, the mammal can achieve an improvement in Index Treatment and Control Lesion by modified Clinical Assessment Scale of Severity for Index Lesion Signs and Symptoms (mCAILS) score from baseline. In some cases, the improvement is statistically significant. In some cases, the mammal can achieve a statistically significant reduction in mCAILS score after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a reduction in the percentage of the Body Surface Area of the cutaneous LP involvement. In some cases, the reduction is statistically significant. In some cases, the mammal can achieve a statistically significant reduction in percentage of the Body Surface Area of the cutaneous LP involvement after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a Physician Global Cutaneous LP Assessment of 0 (clear), 1 (almost clear), or 2 (significant improvement). In some cases, the mammal can achieve a Physician Global Cutaneous LP Assessment of 0 (clear), 1 (almost clear), or 2 (significant improvement) after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a statistically significant improvement in Physician Global Cutaneous LP Assessment from baseline. In some cases, the mammal can achieve a statistically significant improvement in Physician Global Cutaneous LP Assessment from baseline after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with the JAK inhibitor (e.g., baricitinib).

In some cases, the mammal can achieve a 1-point improvement from baseline in Physician Global Cutaneous LP Assessment. In some cases, the mammal can achieve a 1-point improvement from baseline in Physician Global Cutaneous LP Assessment after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib). In some cases, the mammal can achieve a 2-point improvement from baseline in Physician Global Cutaneous LP Assessment. In some cases, the mammal can achieve a 2-point improvement from baseline in Physician Global Cutaneous LP Assessment 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a reduction in mucosal lichen planus, e.g., oral or extraoral LP, such as the vaginal mucosa, from baseline. In some cases, the reduction is statistically significant. In some cases, the mammal can achieve a reduction in at least one of the: Thongprasom score, the Oral Disease Severity Score (ODSS), and/or the REU (Reticular/hyperkeratotic, Erosive/erythematous, Ulcerative) scoring systems, with a decrease in the score from baseline after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib). See, e.g., Unnikrishnan, et al., Disease severity scoring systems in mucosal lichen planus: A systematic review, Oral Diseases, 20 Nov. 2022, doi.org/10.1111/odi.14447, relevant portions incorporated herein by reference.

In some cases, the mammal can achieve a reduction in pruritus Numerical Rating Scale score from baseline. In some cases, the reduction is statistically significant. In some cases, the mammal can achieve a reduction in pruritus Numerical Rating Scale score from baseline after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a reduction in Skindex-16 score from baseline. In some cases, the reduction is statistically significant. In some cases, the mammal can achieve a reduction in Skindex-16 score from baseline after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal has clinical and histological features of LP.

In some cases, the mammal has a Body Surface Area of LP involvement of 2% to 20% at baseline. In some cases, the mammal has a Body Surface Area of LP involvement of 3% to 20% at baseline. In some cases, the mammal has a Body Surface Area of LP involvement in a range of 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 8% to 8%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 20%, 10% to 19%, 10% to 18%, 10% to 17%, 10% to 16%, 10% to 15%, 10% to 14%, 10% to 13%, 10% to 12%, 10% to 11%, 11% to 20%, 11% to 19%, 11% to 18%, 11% to 17%, 11% to 16%, 11% to 15%, 11% to 14%, 11% to 13%, 11% to 12%, 12% to 20%, 12% to 19%, 12% to 18%, 12% to 17%, 12% to 16%, 12% to 15%, 12% to 14%, 12% to 13%, 13% to 20%, 13% to 19%, 13% to 18%, 13% to 17%, 13% to 16%, 13% to 15%, 13% to 14%, 14% to 20%, 14% to 19%, 14% to 18%, 14% to 17%, 14% to 16%, 14% to 15%, 15% to 20%, 15% to 19%, 15% to 18%, 15% to 17%, 15% to 16%, 16% to 20%, 16% to 19%, 16% to 18%, 16% to 17%, 17% to 20%, 17% to 19%, 17% to 18%, 18% to 20%, 18% to 19%, or 19% to 20%.

In some cases, the mammal has a minimum of 4 (e.g., 5, 6, 7, 8, 9, 10, or more than 10) lesions of LP. In some cases, the mammal has a minimum of 10 lesions of LP. In some cases, the mammal has a minimum of 20 lesions of LP. In some cases, the mammal has a minimum of 30 lesions of LP. In some cases, the mammal has a minimum of 40 lesions of LP. In some cases, the mammal has a minimum of 50 lesions of LP. In some cases, the mammal has a minimum of 60 lesions of LP. In some cases, the mammal has a minimum of 70 lesions of LP. In some cases, the mammal has had LP for at least 8 months.

In some cases, the mammal (e.g., a human) has failed to achieve an adequate response to prior therapy for LP. In some cases, the prior therapy is administration of one of the following: one or more topical corticosteroids selected from the group of triamcinolone acetonide, clobetasol propionate, diflorasone diacetate, fluocinolone acetonide, betamethasone valerate, betamethasone dipropionate, amcinonide, desoximetasone, fluocinonide acetonide, halcinonide, hydrocortisone valerate, desonide, hydrocortisone acetate, methylprednisolone acetate, dexamethasone sodium phosphate; topical tacrolimus and pimecrolimus; systemic immunosuppressant; oral metronidazole; oral sulfasalazine; and oral retinoid. In some cases, the prior therapy is administration of one of the following: one or more topical corticosteroids selected from the group of triamcinolone acetonide, clobetasol propionate, diflorasone diacetate, fluocinolone acetonide, betamethasone valerate, betamethasone dipropionate, amcinonide, desoximetasone, fluocinonide acetonide, and halcinonide; topical tacrolimus and pimecrolimus; systemic immunosuppressant; oral metronidazole; oral sulfasalazine; oral retinoid. In some cases, the prior therapy phototherapy. In some cases, the prior therapy is administration of mycophenolate mofetil, methotrexate, azathioprine, or cyclosporine, or a combination thereof.

In some cases, the mammal can achieve a statistically significant reduction in the total lesion count from baseline. For example, the mammal can achieve a statistically significant reduction in the total lesion count from baseline to week 4 of the administering, from baseline to week 8 of the administering, from week 4 to week 8 of the administering, or from week 8 to week 12 of the administering.

In some cases, the mammal can achieve a reduction of at least 1 lesion. For example, the mammal can achieve a reduction of at least 2 lesions, a reduction of at least 3 lesions, a reduction of at least 4 lesions, a reduction of at least 5 lesions, a reduction of at least 6 lesions, a reduction of at least 7 lesions, a reduction of at least 8 lesions, a reduction of at least 9 lesions, or a reduction of at least 10 lesions.

In some cases, the mammal can achieve a reduction of at least 10 lesions in the total lesion count from baseline to week 4 of the administering, or from baseline to week 8 of the administering. In some cases, the mammal can achieve a reduction of at least 10 lesions in the total lesion count from week 4 to week 8 of the administering, or from week 8 to week 12 of the administering.

In some cases, the mammal can achieve a reduction of at least 20 lesions. For example, the mammal can achieve a reduction of at least 20 lesions in the total lesion count from baseline to week 4 of the administering, from baseline to week 8 of the administering, from week 4 to week 8 of the administering, or from week 8 to week 12 of the administering.

In some cases, the mammal achieves a reduction of at least 30 lesions. For example, the mammal can achieve a reduction of at least 30 lesions in the total lesion count from baseline to week 4 of the administering, from baseline to week 8 of the administering, from week 4 to week 8 of the administering, or from week 8 to week 12 of the administering.

In some cases, the mammal can achieve a reduction of at least 40 lesions. For example, the mammal can achieve a reduction of at least 40 lesions in the total lesion count from baseline to week 4 of the administering, from baseline to week 8 of the administering, from week 4 to week 8 of the administering, or from week 8 to week 12 of the administering.

In some cases, the mammal can achieve a reduction of at least 50 lesions. For example, the mammal can achieve a reduction of at least 50 lesions in the total lesion count from baseline to week 4 of the administering, from baseline to week 8 of the administering, from week 4 to week 8 of the administering, or from week 8 to week 12 of the administering.

In some cases, the mammal can achieve a statistically significant reduction in modified Clinical Assessment Scale of Severity for Index Lesion Signs and Symptoms score for treatment lesion from baseline. For example, the mammal can achieve a statistically significant reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 1-point reduction in modified Clinical Assessment Scale of Severity for Index Lesion Signs and Symptoms score for treatment lesion from baseline. For example, the mammal can achieve at least a 1-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 2-point reduction in modified Clinical Assessment Scale of Severity for Index Lesion Signs and Symptoms score for treatment lesion from baseline. For example, the mammal can achieve at least a 2-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 3-point reduction in modified Clinical Assessment Scale of Severity for Index Lesion Signs and Symptoms score for treatment lesion from baseline. For example, the mammal can achieve at least a 3-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 4-point reduction in modified Clinical Assessment Scale of Severity for Index Lesion Signs and Symptoms score for treatment lesion from baseline. For example, the mammal can achieve at least a 4-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 5-point reduction in modified Clinical Assessment Scale of Severity for Index Lesion Signs and Symptoms score for treatment lesion from baseline. For example, the mammal can achieve at least a 5-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 6-point reduction in modified Clinical Assessment Scale of Severity for Index Lesion Signs and Symptoms score for treatment lesion from baseline. For example, the mammal can achieve at least a 6-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve at least a 7-point reduction in modified Clinical Assessment Scale of Severity for Index Lesion Signs and Symptoms score for treatment lesion from baseline. For example, the mammal can achieve at least a 7-point reduction in modified Clinical Assessment Scale of Severity for Index Lesion Signs and Symptoms score for treatment lesion from baseline to week 4 of the administering, from baseline to week 8 of the administering, from week 4 to week 8 of the administering, or from week 8 to week 12 of the administering.

In some cases, the mammal can achieve a reduction in the percentage of the Body Surface Area of the cutaneous LP involvement. In some cases, the reduction can be statistically significant. For example, the mammal can achieve a statistically significant reduction in the percentage of the Body Surface Area score of the cutaneous LP involvement from baseline to week 4 of the administering, from baseline to week 8 of the administering, from week 4 to week 8 of the administering, or from week 8 to week 12 of the administering.

In some cases, the mammal can achieve at least a 10% reduction in the percentage of the Body Surface Area of the cutaneous LP involvement. In some cases, the reduction in percentage of the Body Surface Area of the cutaneous LP involvement can be achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 20% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 30% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 40% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 50% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 60% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 70% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 80% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve at least a 90% reduction in the percentage of the Body Surface Area of the cutaneous LP involvement. In some cases, the reduction in the percentage of the Body Surface Area of the cutaneous LP involvement is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve Physician Global Cutaneous LP Assessment of 0 (clear), 1 (almost clear), or 2 (significant improvement). In some cases, the Physician Global Cutaneous LP Assessment of 0 (clear), 1 (almost clear), or 2 (significant improvement) is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve Physician Global Cutaneous LP Assessment of 0 (clear). In some cases, the Physician Global Cutaneous LP Assessment of 0 (clear) is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve Physician Global Cutaneous LP Assessment of 1 (almost clear). In some cases, the Physician Global Cutaneous LP Assessment of 1 (almost clear) is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve Physician Global Cutaneous LP Assessment of 2 (significant improvement). In some cases, the Physician Global Cutaneous LP Assessment of 2 (significant improvement) is achieved after 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks of treatment with a JAK inhibitor (e.g., baricitinib).

In some cases, the mammal can achieve a reduction in pruritus Numerical Rating Scale score from baseline. In some cases, the reduction is statistically significant. For example, the mammal can achieve a statistically significant reduction in in pruritus Numerical Rating Scale score from baseline to week 4 of the administering from baseline to week 8 of the administering, from week 4 to week 8 of the administering, or from week 8 to week 12 of the administering.

In some cases, the mammal can achieve at least a 1-point reduction in pruritus Numerical Rating Scale score from baseline. For example, the mammal can achieve at least a 1-point reduction in in pruritus Numerical Rating Scale score from baseline to week 4 of the administering, from baseline to week 8 of the administering, from week 4 to week 8 of the administering, or from week 8 to week 12 of the administering.

In some cases, the mammal can achieve at least a 2-point reduction in pruritus Numerical Rating Scale score from baseline. For example, the mammal can achieve at least a 2-point reduction in in pruritus Numerical Rating Scale score from baseline to week 4 of the administering, from baseline to week 8 of the administering, from week 4 to week 8 of the administering, or from week 8 to week 12 of the administering.

In some cases, the mammal can achieve at least a 3-point reduction in pruritus Numerical Rating Scale score from baseline. For example, the mammal can achieve at least a 3-point reduction in in pruritus Numerical Rating Scale score from baseline to week 4 of the administering, from baseline to week 8 of the administering, from week 4 to week 8 of the administering, or from week 8 to week 12 of the administering.

In some cases, the mammal can achieve at least a 4-point reduction in pruritus Numerical Rating Scale score from baseline. For example, the mammal can achieve at least a 4-point reduction in in pruritus Numerical Rating Scale score from baseline to week 4 of the administering, from baseline to week 8 of the administering, from week 4 to week 8 of the administering, or from week 8 to week 12 of the administering.

In some cases, the mammal can achieve a reduction in the Skindex-16 score. In some cases, the reduction is statistically significant. For example, the mammal can achieve a statistically significant reduction in Skindex-16 score from baseline to week 4 of the administering, from baseline to week 8 of the administering, from week 4 to week 8 of the administering, or from week 8 to week 12 of the administering.

In some cases, the mammal can achieve at least a 10-point reduction in the Skindex-16 score. For example, the mammal can achieve at least a 10-point reduction in Skindex-16 score from baseline to week 4 of the administering, from baseline to week 8 of the administering, from week 4 to week 8 of the administering, or from week 8 to week 12 of the administering.

In some cases, the mammal can achieve at least a 20-point reduction in the Skindex-16 score. For example, the mammal can achieve at least a 20-point reduction in Skindex-16 score from baseline to week 4 of the administering, from baseline to week 8 of the administering, from week 4 to week 8 of the administering, or from week 8 to week 12 of the administering.

In some cases, the mammal can achieve at least a 30-point reduction in the Skindex-16 score. For example, the mammal can achieve at least a 30-point reduction in Skindex-16 score from baseline to week 4 of the administering, from baseline to week 8 of the administering, from week 4 to week 8 of the administering, or from week 8 to week 12 of the administering.

In some cases, the mammal can achieve at least a 40-point reduction in the Skindex-16 score. For example, the mammal can achieve at least a 40-point reduction in Skindex-16 score from baseline to week 4 of the administering, from baseline to week 8 of the administering, from week 4 to week 8 of the administering, or from week 8 to week 12 of the administering.

The treatment can be carried out for any appropriate length of time. For example, the administering can be maintained for at least 4 weeks (e.g., at least 8 weeks or at least 12 weeks). In some cases, the oral formulation can be administered twice per day, where the two administrations are at least 8 hours apart (e.g., about 12 hours apart).

In some cases, the methods provided herein can further include administering to the mammal an additional therapeutic agent.

This document provides, inter alia, methods of treating cutaneous LP in a mammal in need thereof. The methods can include administering, to a mammal having LP, an oral formulation containing a JAK inhibitor. In some cases, the mammal can be a man or woman at least 18 years of age. The mammal can have clinical and/or histological features of LP. For example, the mammal can have a Body Surface Area of LP involvement of 2% to 20% at baseline, a minimum of 4 lesions of LP, a minimum of 10 lesions of LP, or a combination thereof. In some cases, the mammal can achieve a reduction of at least 50 lesions in total lesion count from baseline after treatment with an orally administered JAK inhibitor.

As used herein, “CAILS” refers to clinical assessment scale of severity for index lesion signs and symptoms, which has a 9-point scale as shown TABLE 1:

TABLE 1

| Score | Grade |
|---|---|
| 0 | No evidence of sign or symptom |
| 1 | Intermediate interval |
| 2 | Mild: less than average presentation of sign or symptom |
| 3 | Intermediate interval |
| 4 | Moderate: average disease presentation of sign or symptom |
| 5 | Intermediate interval |
| 6 | Severe: greater than 25% worse than average severity of |
| | sign or symptom |
| 7 | Intermediate interval |
| 8 | Very severe: the near worst severity sign or symptom |

The term “modified CAILS” or “mCAILS” score as used herein refers to summation of Erythema (0-8), Scaling (0-8), Plaque elevation (0-8), and Size (0-18). The modified CAILS score range is from 0 to 42, and higher scores indicate higher severity. A scale of 0 to 18 is used to grade lesion size by square centimeter (0, no measurable area; 1, >0 cm² and <4 cm²; 2, >4 cm² and <10 cm²; 3, >10 cm² and <16 cm²; 4, >16 cm² and <25 cm²; 5, >25 cm² and <35 cm²; 6, >35 cm² and <45 cm², 7, >45 cm² and <55 cm²; 8, >55 cm² and <70 cm²; 9, >70 cm² and <90 cm²; 10, >90 cm² and <110 cm²; 11, >110 cm² and <130 cm²; 12, >130 cm² and <155 cm²; 13, >155 cm² and <180 cm²; 14, >180 cm² and <210 cm²; 15, >210 cm² and <240 cm²; 16, >240 cm² and <270 cm²; 17, >270 cm² and <300 cm²; and 18, >300 cm²). The area of a lesion can be measured with, for example, digital planimetry (see, e.g., Olsen et al., J Clin Oncol, 29(18): 2598-2607, 2011; Duvic et al., Arch Dermatol, 137(5):581-593, 2001; and Rogers et al., J Diabetes Sci Technol, 4(4): 799-802, 2010).

“PGA” refers to Physician Global Assessment. The PGA can be used to evaluate the severity of cutaneous LP, and also is referred to as the Physician Global Cutaneous LP Assessment (PhCLPGA), which has a 7-point scale as shown in TABLE 2:

TABLE 2

| Score | Severity | Description |
|---|---|---|
| 0 | Clear | No evidence of disease (100% improvement) CCR |
| 1 | Almost clear | Very significant clearance (≥90% to 100%) PR |
| 2 | Marked improvement | Significant improvement (≥75% to <90%) PR |
| 3 | Moderate improvement | Intermediate between slight and marked (≥50% to <75%) PR |
| 4 | Slight improvement | Some improvement (≥25% to <50%) but significant evidence of |
| | | disease remains SD |
| 5 | No change | Disease has not changed from baseline condition (±<25%) SD |
| 6 | Worse | Disease is worse than at baseline evaluation (≥25%) or more PD |

CCR: Complete clinical response
PR: Partial response
SD: Stable disease
PD: Progressive Disease

“Pruritus NRS score” or “itch NRS score” refers to pruritus Numerical Rating Scale. The pruritus NRS is a daily patient-reported measure (24-hour recall) of itch intensity. Subjects are be asked to rate the itching severity due to their LP by selecting a number from 0 (no itch) to 10 (worst imaginable itch) as shown in TABLE 3 below that best describes their worst level of itching in the past 24 hours. In some cases, a subject can be issued a hand-held device (eDiary) on which to record itch severity, and may be instructed to complete the eDiary each night.

TABLE 3

| Pruritus NRS score | Itchiness |
|---|---|
| 0 | No itch |
| 1-4 | 1-4 Mild itch |
| 4-7 | 4-7 Moderate itch |
| 7-9 | 7-9 Severe itch |
| 10 | 10 Very severe itch |

A “Skindex 16” score refers to the total Skindex-16 score. Skindex 16 is a 16-item patient-completed assessment to rate skin conditions using numerical analogue scales (0=never bothered to 6=always bothered). Responses to the Skindex-16 are categorized into three subscales: symptom, emotional, and functional as shown in TABLE 4 below. The total Skindex-16 score is 0-96.

TABLE 4

| Subscale | | |
|---|---|---|
| Categories | Subscale (0 = never bothered to 6 = always bothered) | |
| Symptom | 1. | Skin itching |
| subscale | 2. | Skin burning or stinging |
| | 3. | Skin hurting |
| | 4. | Skin irritated |
| Emotional | 5. | Persistence or recurrence of condition |
| subscale | 6. | Worry about condition |
| | 7. | Appearance of skin |
| | 8. | Frustration about skin |
| | 9. | Embarrassment about skin |
| | 10. | Annoyed about skin |
| | 11. | Feeling depressed |
| Functional | 12. | Effect of skin on interaction with others |
| subscale | 13. | Effect of skin on desire to be with people |
| | 14. | Skin making it hard to show affection |
| | 15. | Effect of skin on daily activity |
| | 16. | Skin making it hard to work/have enjoyment |

The methods provided herein can included orally administering a composition containing a JAK inhibitor and a pharmaceutically acceptable carrier. As used herein, the phrase “pharmaceutically acceptable” refers to those compounds, materials, compositions, and/or dosage forms, which are, within the scope of sound medical judgment, suitable for use in contact with tissues of humans and animals. In some cases, “pharmaceutically acceptable” refers to compounds, materials, compositions, and/or dosage forms approved by a regulatory agency of the Federal or a state government or listed in the U.S. Pharmacopeia or other generally recognized pharmacopeia for use in animals, and more particularly in humans.

As used herein, the phrase “pharmaceutically acceptable” means those compounds, materials, compositions, and/or dosage forms, which are, within the scope of sound medical judgment, suitable for use in contact with tissues of humans and animals. In some cases, “pharmaceutically acceptable” means approved by a regulatory agency of the Federal or a state government or listed in the U.S. Pharmacopeia or other generally recognized pharmacopeia for use in animals, and more particularly in humans.

The present invention also includes pharmaceutically acceptable salts of the JAK inhibitors that can be orally administered as described herein. As used herein, “pharmaceutically acceptable salts” refers to derivatives of the disclosed compounds in which the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile (MeCN) can be particularly useful. Lists of suitable salts are found in Remington's Pharmaceutical Sciences, 17th ed., Mack Publishing Company, Easton, Pa., 1985, p. 1418; and J Pharm Sci, 66:2, 1977), each of which is incorporated herein by reference in its entirety. In some cases, the pharmaceutically acceptable salt can be a phosphoric acid salt, a sulfuric acid salt, or a maleic acid salt.

In some cases, the compounds, or pharmaceutically acceptable salts thereof, or pharmaceutical formulations thereof, oral formulations thereof, as described herein are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; or (3) preventing the disease, condition or disorder in an individual who may be predisposed to the disease, condition or disorder but does not yet experience or display the pathology or symptomatology of the disease. In some cases, treating refers to inhibiting or ameliorating the disease. In some cases, treating is preventing the disease. In some cases, the components are present in exactly the ranges specified (e.g., the term “about” is not present). In some cases, “about” means plus or minus 10% of the value.

The methods described herein can further include administering one or more additional therapeutic agents. One or more additional therapeutic agents can be administered to a patient simultaneously or sequentially.

In some cases, the additional therapeutic agent is an antibiotic. In some cases, the antibiotic is clindamycin, doxycycline, minocycline, trimethoprimsulfamethoxazole, erythromycin, metronidazole, rifampin, moxifloxacin, dapsone, or a combination thereof. In some cases, the antibiotic is clindamycin, doxycycline, minocycline, trimethoprim-sulfamethoxazole, or erythromycin in combination with metronidazole. In some cases, the antibiotic is a combination of rifampin, moxifloxacin, and metronidazole. In some cases, the antibiotic is a combination of moxifloxacin and rifampin.

In some cases, the additional therapeutic agent is a retinoid (e.g., adapalene, etretinate, acitretin, or isotretinoin).

In some cases, the additional therapeutic agent is a steroid. In some cases, the additional therapeutic agent is a corticosteroid. In some cases, the steroid is such as triamcinolone, dexamethasone, fluocinolone, cortisone, prednisone, prednisolone, or flumetholone.

In some cases, the additional therapeutic agent is an immunosuppressant. In some cases, the immunosuppressant is methotrexate or cyclosporin A. In some cases, the immunosuppressant is mycophenolate mofetil or mycophenolate sodium.

In some cases, the additional therapeutic agent is azelaic acid.

This document also includes pharmaceutical kits that can be used, for example, in the treatment and/or prevention of LP. The kits can include one or more containers containing a pharmaceutical composition comprising a therapeutically effective amount of a JAK inhibitor or a pharmaceutically acceptable salt thereof, as described herein. Such kits can further include, if desired, one or more of various conventional pharmaceutical kit components, such as one or more containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be further described in the following example, which does not limit the scope of the invention described in the claims.

### EXAMPLE

A clinical study was conducted in which 12 patients with biopsy-proven LP were trialed on oral baricitinib at a daily dose of 2 mg for 16 weeks. Other inclusionary criteria included having at least 4 lesions of LP at baseline. Patient demographics are listed in TABLE 5. The mean age of participants was 63.6 years. The vast majority were female and identified as Caucasian. A quarter were under-represented minorities. The mean duration of disease for all patients was 2.2 years. Five of the 12 participants had hypertrophic LP, and 11 were refractory to prior therapy. All but one patient had previously trialed topical steroids. Almost half (41.7%) had failed oral or IM steroids. One patient had failed methotrexate and one had failed to respond to topical calcineurin inhibitors.

TABLE 5

| | |
|---|---|
| Age (mean, SD), y | 63.6 (13.6) |
| Sex (female) | 11 (91.7%) |
| Race | |
| Black | 1 (8.3%) |
| Caucasian | 9 (75.0%) |
| Hispanic | 2 (16.7%) |
| Duration of disease (mean) | 2.2 years |
| Hypertrophic LP | 5 (41.7%) |
| Prior therapies | |
| Topical steroids | 11 (91.7%) |
| Oral or IM steroids | 5 (41.7%) |
| Methotrexate | 1 (8.3%) |
| Topical calcineurin | 1 (8.3%) |

The primary outcome of this study was PGA at week 16, with treatment response defined as greater than 50% improvement with a PGA grade of 0 to 3. Non-responsive was defined as PGA 4 and 5. Secondary endpoints included changes from baseline to week 16 in mCAILS, pruritus scores, Skindex-16, BSA, and total lesion count.

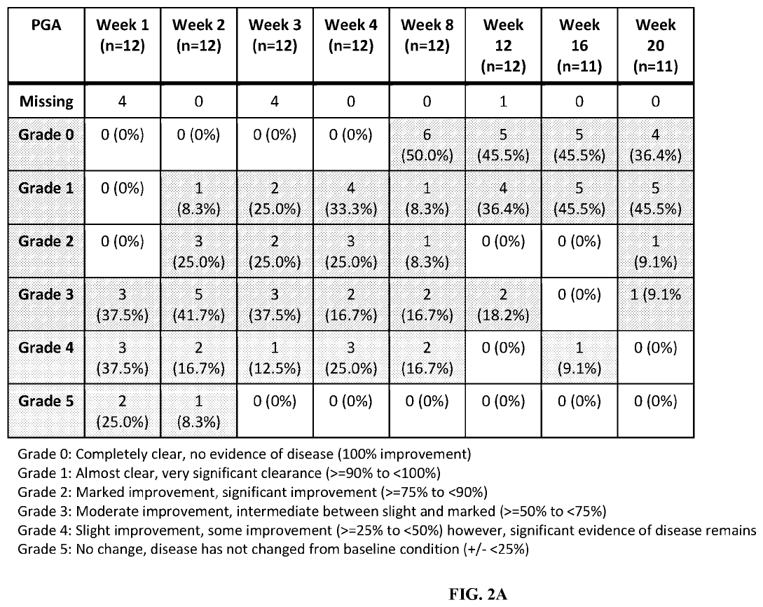

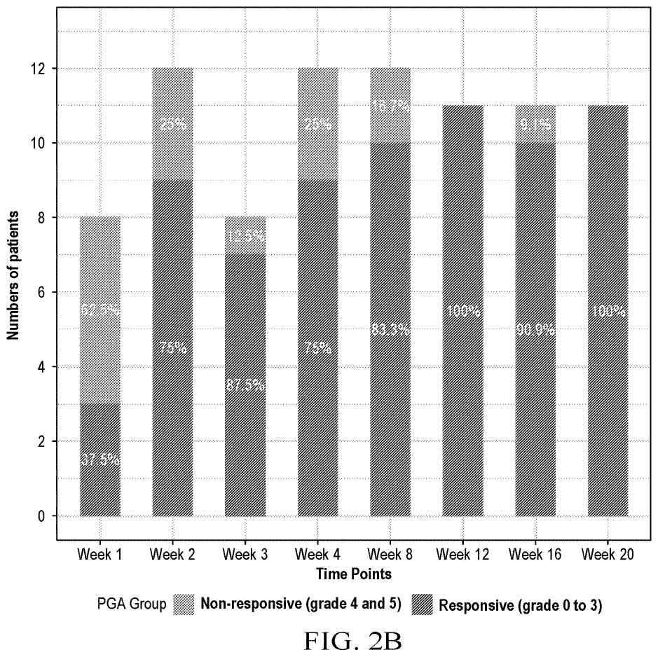

Treatment response was seen at week 1 in 37.5% of patients, and 100% of patients were responsive at week 12 (FIGS. 2A and 2B). At week 16 (the primary endpoint), all but one patient (90.9%) had a PGA grade of 0 or 1 (corresponding to completely clear and almost clear), which indicated a dramatic response to baricitinib. Treatment effects were sustained at week 20 (after 4 weeks of being off-therapy), with 100% response.

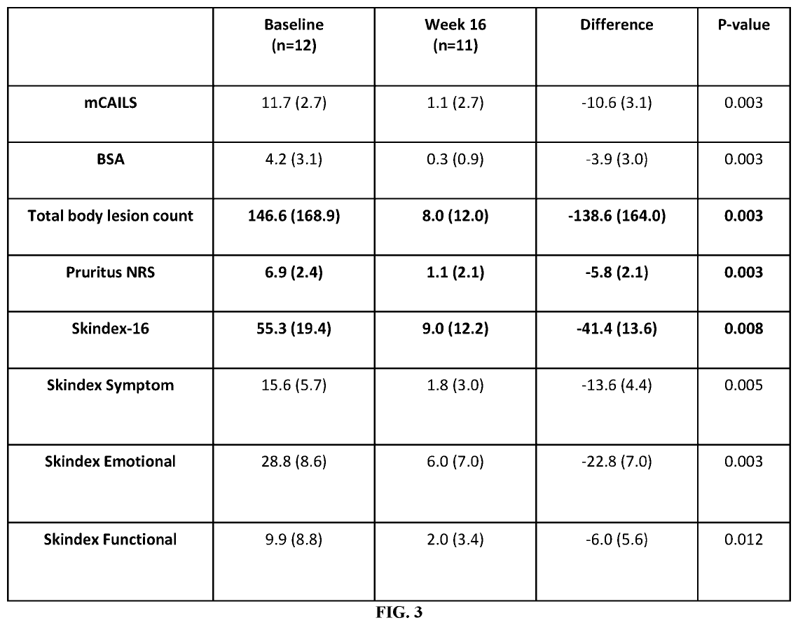

Improvements also were observed across all secondary measures (FIG. 3). The mean total body lesion count was 146 at baseline, and it dropped to 8 by week 16. Quality of life indices also improved. At the beginning of the trial, the QoL and Pruritus NRS scores were similar to severe AD. For example, the average pruritus score was about 7 at the start of trial, which would be categorized as severe pruritus. By week 16, there was a dramatic improvement in itch, with barely perceptible itch—a dramatic and clinically meaningful response. As a reference, a meaningful improvement in itch was defined as an improvement of 4 points or greater that was sustained. A total of 12 adverse events also were observed, but only one (a low neutrophil count that was not deemed serious) appeared to be related to the study medication. The remaining 11 adverse events were unrelated or unlikely to be related to baricitinib.

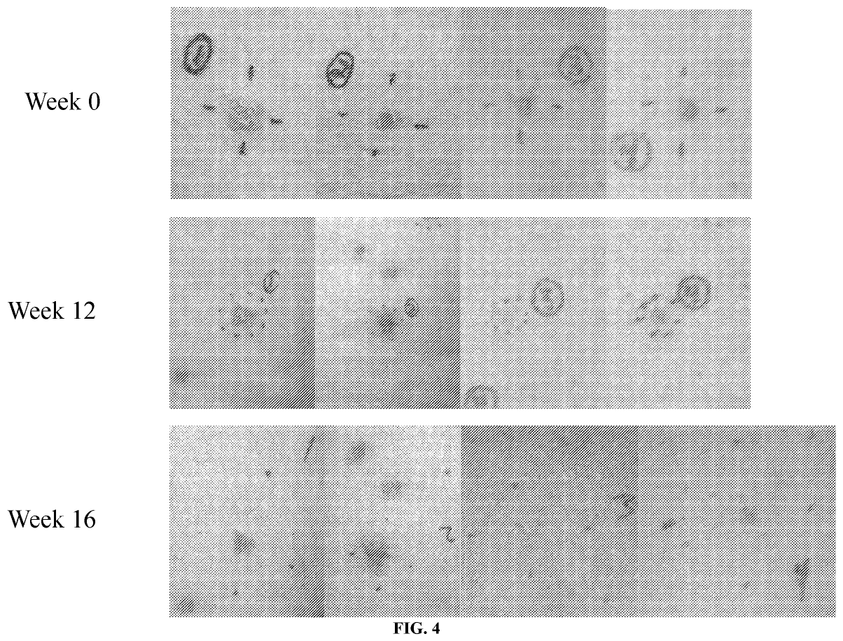

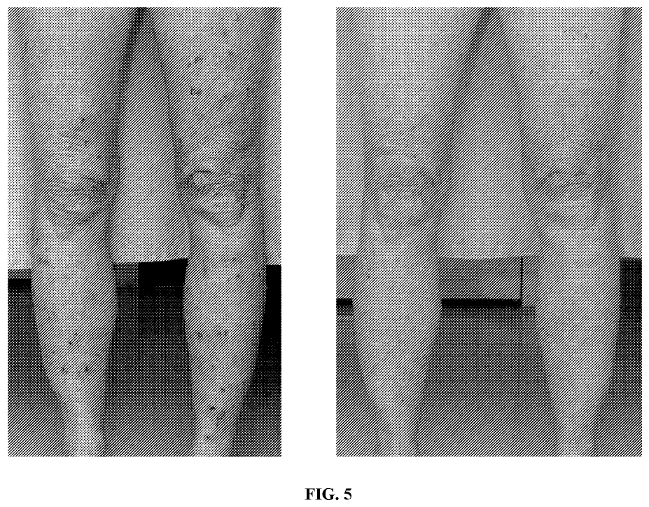

Representative pre- and post-treatment images are shown in FIG. 4, comparing weeks 0, 12, and 16. These images clearly show response to baricitinib treatment. Thus, the studies described herein showed that oral baricitinib demonstrated a rapid disease response, as early as week 1, with 100% response at Week 12 and Week 20, improvement across all disease and QoL metrics. FIG. 5 shows image of cutaneous LP response to baricitinib (Day 0 vs. Week 16).

### Baricitinib in the Treatment of Cutaneous Lichen Planus.

The present example provides the results of an open-label, single-arm study of baricitinib in cutaneous LP and defined the molecular profile and signature of disease using bulk, spatial, and single cell RNA sequencing on pre- and post-treatment specimens.

Trial Design. A single-arm, open-label, phase 2, first-in-human trial was conducted at Mayo Clinic, Arizona (ClinicalTrials.gov identifier NCT05188521). The Mayo Clinic Institutional Review Board approved the study (IRB 21-003075), and all patients provided written informed consent. Twelve patients with biopsy-proven cutaneous LP were administered oral baricitinib 2 mg once daily for 16-weeks. The primary endpoint was an overall response by Physician Global Assessment (PGA) of skin at week 16, with treatment response defined as PGA 0 to 3 (with ≥50% score reduction). Secondary outcomes were changes in mCAILS, total body lesion count, affected BSA, pruritus numeric rating scale (NRS), pruritus verbal rating scale (VRS), pruritus visual analog scale (VAS), pain NRS, and Skindex-16. Patients were evaluated at baseline (week 0) and weeks 2, 4, 8, 12, and 16. Treatment-responsive patients who did not achieve PGA grade 0 at week 16 were eligible to enroll in the dose escalation for an additional 12 weeks of treatment with oral baricitinib 4 mg daily. Complete responders at week 16 were reassessed at week 20 and partial responders at week 16 were reassessed at week 32, after an off-therapy period of 4 weeks, respectively.

Assessments of treatment efficacy, adverse events (AEs), and QoL were completed at each study visit. All lesions were annotated, photographed, and scored using the mCAILS criteria. 16 All clinical assessments were performed by the principal investigator and co-investigators (ARM, MRP, SZ) with questionable lesions scored by 2 investigators. The revised National Cancer Institute Common Terminology Criteria for Adverse Events version 5.0 was used for AE reporting. Due to travel issues, one patient withdrew from the study after week 8. An intention-to-treat (ITT) analysis was used with the patient's last observation to impute week 16 data.

Skin biopsies were collected at baseline and week 2 of LP lesional skin and normal appearing skin for bulk RNA-sequencing. Week 2 samples were designated as responsive (defined as a lesion with >50% response by mCAILS) or nonresponsive (defined as a lesion with <50% response by mCAILS). Additional 6-8 mm biopsies of lesional tissue were taken at weeks 0 and 2 for spatial sequencing and scRNA-seq, and blood for scRNA-seq.

Eligibility Criteria. Patients aged ≥18 years with biopsy-proven cutaneous LP were eligible for the trial. Both treatment-naïve and treatment-refractory disease were included. Key exclusion criteria included predominantly non-cutaneous variants of LP, active infections, and other active inflammatory cutaneous conditions.

Statistical Analysis. Patient demographics, clinical characteristics, and outcomes were summarized as mean, standard deviation, median, interquartile range for continuous variables, and frequency and percentages for categorical variables. Primary and secondary outcome differences between baseline and week 16 were compared using Wilcoxon signed rank test for continuous variables and McNemar's test for binary variables. One patient withdrew from the study after week 8, and this patient's last observation (week 8) was used to impute week 16 data in the ITT analysis.

Exact binomial method was used to calculate the treatment response rate at week 16 and its corresponding 95% confidence interval. All analyses were conducted with R version 4.1.2 (R Foundation for Statistical Computing, Vienna, Austria). The cut-off for statistical significance was set as 0.05.

Transcriptomic Analyses. Bulk RNA sequencing, scRNA sequencing, and spatial sequencing were conducted on pre- and post-treatment timepoints on control and disease samples. T-cell receptor gene expression and cell type annotations were conducted on skin samples to identify clonally expanded T-cell types. Immunohistochemical staining of CD3, IFN-γ, CXCL9, and pSTAT2 were performed on skin samples.

Patients. A total of 12 patients with a mean age of 63.6 (SD 13.6) years were enrolled. The majority (n=11, 91.7%) were female and identified as White (n=9, 75.0%). Underrepresented minority patients constituted the remaining 25%. The mean disease duration across all patients was 26.5 months (SD 30.8). All patients had treatment-refractory LP, with 91.7% failing topical steroids, 41.7% failing oral and intramuscular steroids or non-steroidal immunosuppressants, 8.3% failing methotrexate, and 8.3% failing topical calcineurin inhibitors. Classic LP was the predominant form, with hypertrophic LP seen in five (41.7%) patients and mucosal involvement in two subjects (16.7%).

The average affected body surface area (BSA) was 4.9% at baseline (SD 3.7) with a mean of 151.9 total body LP lesions per patient (range 4-600). The mean baseline mCAILS score was 12.3 (SD 3.2) and the overall Skindex-16 was 59.0 (SD 22.1). Pruritus NRS and pruritus VAS scores were 7.2 (SD 2.4) and 6.6 (SD 1.6), respectively, with 91.7% of patients rating their level of itch as moderate/severe on the pruritus VRS. The baseline pain NRS score was 7.7 (SD 1.7).

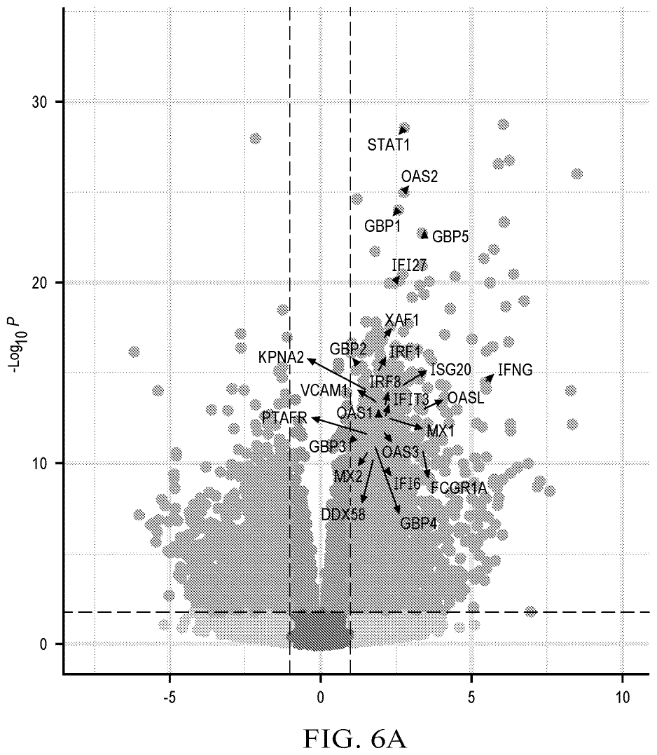

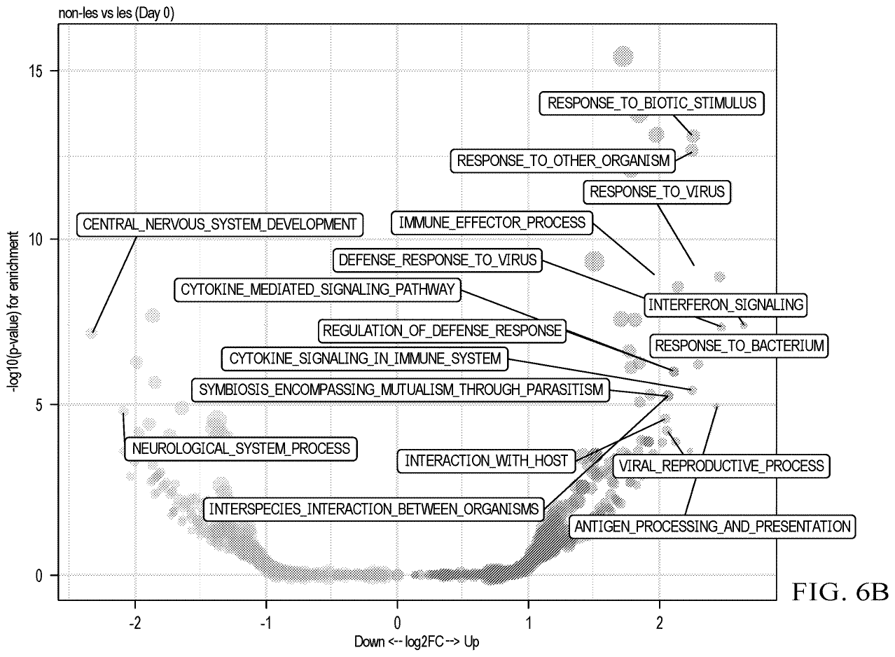

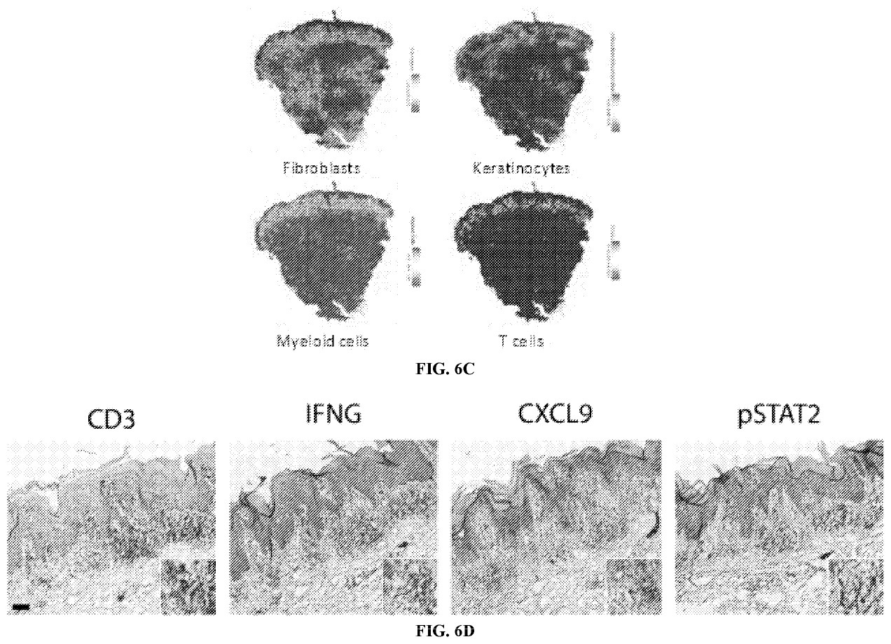

Efficacy. At week 16, 10 of 12 (83.3%; 95% CI: 51.6%-97.9%) patients demonstrated treatment response, achieving PGA scores of 0 to 3, with ≥50% score reduction (FIGS. 6A to 6D; Table 6). Five of the 10 treatment-responsive patients had a PGA of 0 (completely clear), and five had a PGA of 1 (almost clear). Improvement in PGA was observed as early as week 1 in 37.5% of patients and in 100% of patients by week 12. Treatment effects were sustained at week 20 (4 weeks off-therapy), with all patients demonstrating continued response.

TABLE 6
Primary and secondary endpoints at baseline
and week 16 (intention-to-treat analysis)

| PGA | Baseline | Week 16 | Difference | P value |
|---|---|---|---|---|
| Responsive (n, %) | | 83.3% | | |
| Non-responsive (n, %) | | 16.7% | | |
| Total Body Lesion Count | | | | 0.002 |
| Mean (SD) | 151.9 (162.1) | 17.1 (33.5) | −134.8 (157.0) | |
| Range | 4.0-600.0 | 0.0-117.0 | −570.0 to −4.0 | |
| mCAILS | | | | 0.002 |
| Mean (SD) | 12.3 (3.2) | 1.7 (3.3) | −10.6 (2.9) | |
| Range | 7.0-18.6 | 0.0-8.9 | −14.6 to −5.0 | |
| BSA affected (%) | | | | 0.002 |
| Mean (SD) | 4.9 (3.7) | 1.0 (2.5) | −3.9 (2.9) | |
| Range | 0.1-12.0 | 0.0-8.5 | −9.8 to −0.1 | |
| Pruritus NRS | | | | 0.003 |
| Mean (SD) | 7.2 (2.4) | 1.8 (3.2) | −5.3 (2.6) | |
| Range | 1.0-10.0 | 0.0-10.0 | −8.0 to −0.0 | |
| Pruritus VAS | | | | 0.003 |
| Mean (SD) | 6.6 (1.6) | 1.7 (3.0) | −5.0 (2.6) | |
| Range | 2.7-8.8 | 0.0-9.0 | −7.7 to 1.5 | |
| Pain NRS | | | | 0.005 |
| Mean (SD) | 7.7 (1.7) | 1.9 (3.2) | −5.6 (2.7) | |
| Range | 4.0-10.0 | 0.0-10.0 | −8.0 to 0.0 | |
| Skindex-16 overall | | | | 0.008 |
| Mean (SD) | 59.0 (22.1) | 16.9 (28.7) | −37.3(18.3) | |
| Range | 35.0-96.0 | 0.0-96.0 | −58.0 to 0.0 | |
| Skindex-16 symptom | | | | 0.005 |
| Mean (SD) | 16.4 (6.0) | 3.7 (7.0) | −12.4 (5.8) | |
| Range | 4.0-24.0 | 0.0-24.0 | −19.0 to 0.0 | |
| Skindex-16 emotional | | | | 0.003 |
| Mean (SD) | 29.9 (9.0) | 9.0 (12.4) | −20.9 (9.4) | |
| Range | 17.0-42.0 | 0.0-42.0 | −34.0 to 0.0 | |
| Skindex-16 functional | | | | 0.012 |
| Mean (SD) | 11.6 (10.2) | 4.5 (9.0) | −5.5 (5.6) | |
| Range | 0.0-30.0 | 0.0-30.0 | −15.0 to 0.0 | |

Improvements were seen across all secondary measures at week 16 (Table 6). The mean total body lesion count decreased to 17.1 (SD 33.5; p=0.002), and the mean affected BSA decreased to 1.0 (SD 2.5; p=0.002). Compared to a baseline score of 7.2 (SD 2.4), pruritus NRS decreased to 1.8 (SD 3.2; p=0.003), pruritus VAS decreased from 6.6 (SD 1.6) to 1.7 (SD 3.0; p=0.003), and pain NRS decreased from 7.7 (SD 1.7) to 1.9 (SD 3.2; p=0.005). Pruritus NRS improvement >4 from baseline (NRS4) and Pain NRS4 were achieved in 75.0% and 66.7% of patients, respectively. The overall Skindex-16 score decreased from baseline to week 16 by a mean of 37.3 (SD 18.3; p=0.008), accompanied by decreases in each Skindex subscore: Symptom-12.4 (SD 5.8; p=0.005), Emotional-20.9 (SD 9.4; p=0.003), Functional-5.5 (SD 5.6; p=0.012). Results from the per-protocol analysis, with the population defined as patients who completed 16 weeks of baricitinib, were consistent with the results of the ITT analysis.

Dose Escalation. Five of six eligible patients participated in the dose escalation period. All five patients completed an additional 12 weeks of treatment with baricitinib 4 mg daily. At the primary endpoint of week 16, corresponding with the start of dose escalation, 80.0% of patients had PGA grade 1, and 20% had PGA grade 4. After 12 weeks of baricitinib 4 mg daily, 60.0% of patients were completely clear of disease (PGA grade 0), 20.0% were almost clear (PGA grade 1), and 20.0% had slight improvement (PGA grade 4). All but one patient remained treatment-responsive upon re-evaluation after 4 weeks off therapy.

Safety. There were a total of 12 AEs, with only one mild AE that was deemed probably related to the study drug (absolute neutrophil count 0.78×10(9)/L). Most AEs were mild or moderate (58.3% and 25.0%, respectively). No AEs led to the discontinuation of baricitinib.

Molecular Profiling. Whole transcriptomic bulk RNA-seq was performed on lesional and non-lesional skin prior to therapy (n=11, 12, respectively). Analysis for differentially expressed genes (DEGs) (False Discovery Rate, FDR≤0.05, and Fold Change (FC)≥2), revealed a total of 3,524 DEGs, with 1,683 increased and 1,841 decreased compared to non-lesional LP skin at baseline. The most prominent DEGs in lesional LP skin were interferon-stimulated genes (ISGs), including STAT1 (FC-6.9, FDR=8×10-25), OAS2 (FC=6.7, FDR=9×10-22), MX1 (FC=4.8, FDR=1×10-10), and ISG20 (FC=6.6, FDR=2.9×10-12), with IFN-γ (FC=44.6, FDR-2.0×10-12) (FIG. 6A). No differences were observed in the expression of type I IFN genes. Enriched biological processes included, immune-effector process, response to virus, interferon signaling, and antigen processing and presentation (FIG. 6B). Spatial sequencing data from lesional LP showed dense myeloid and T-cells signatures in the upper dermis adjacent to the epidermis (FIG. 6C) and validated by immunohistochemistry (FIG. 6D).

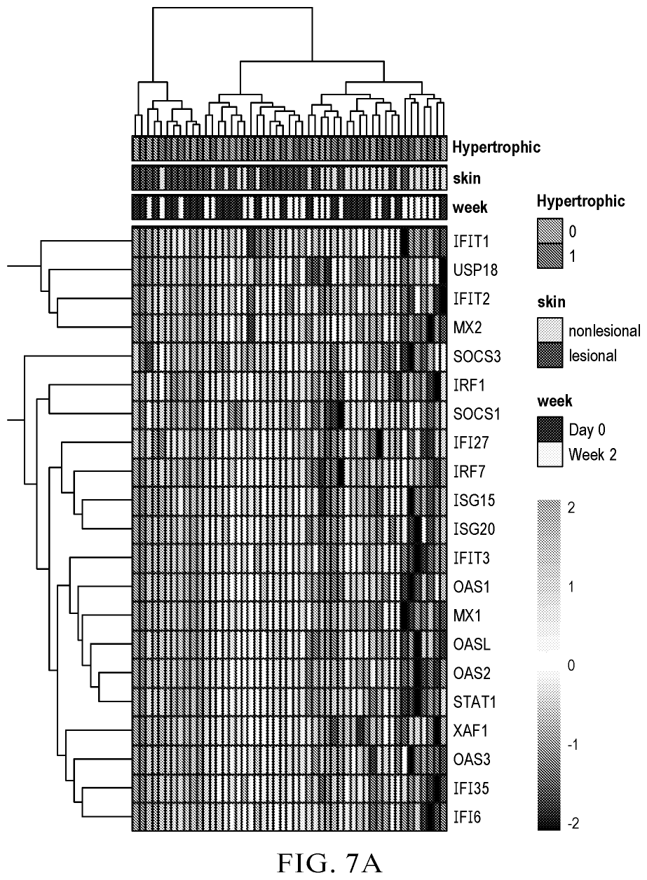

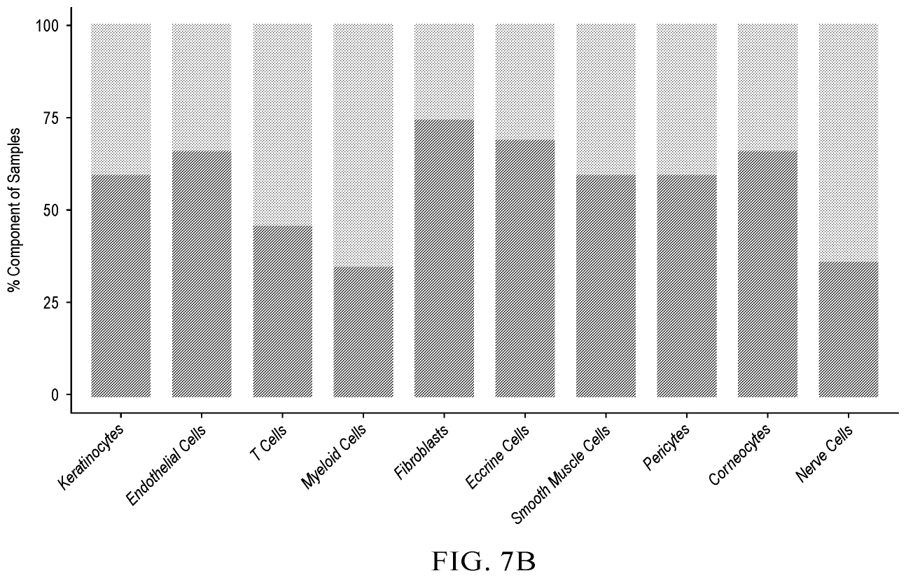

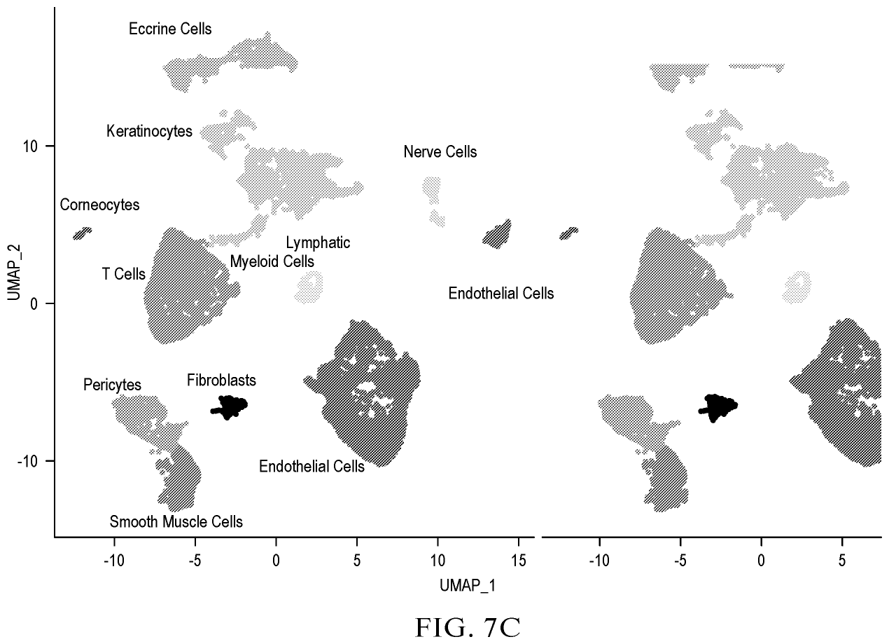

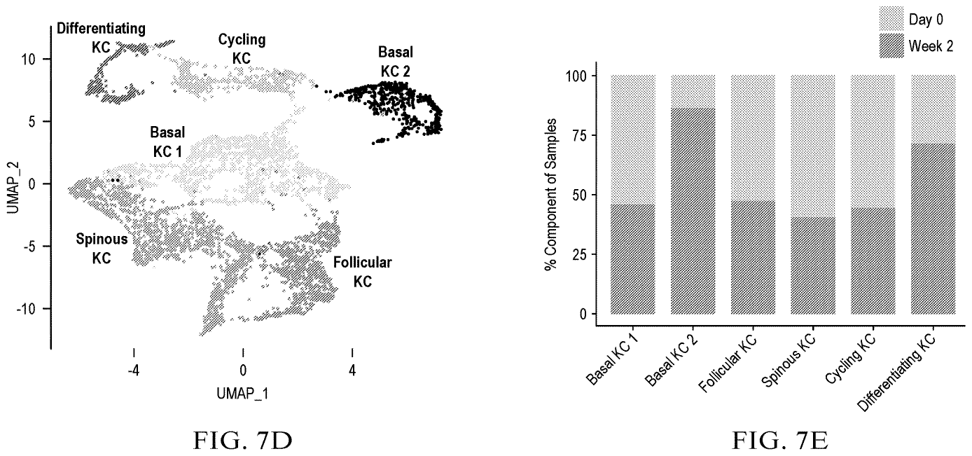

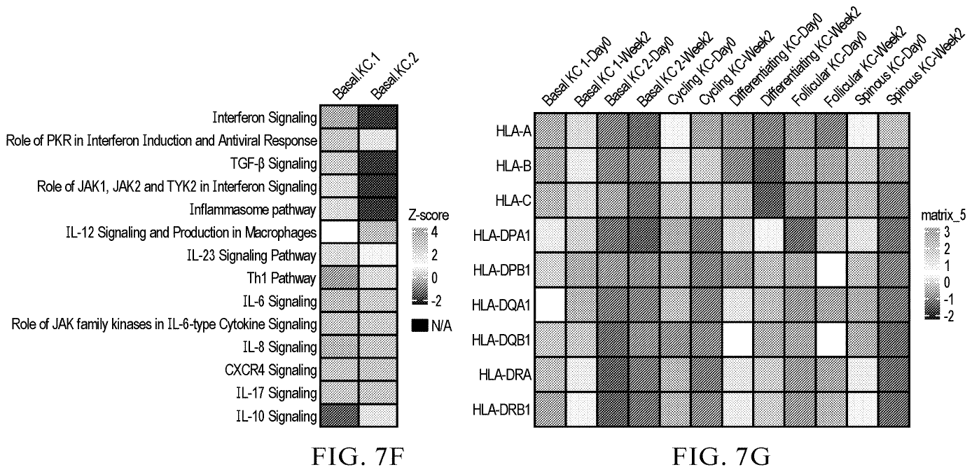

Early tissue transcriptomic response was seen after only two weeks of baricitinib treatment, with marked suppression of interferon stimulated genes (FIG. 7A), and interferon signaling (p=1.2×10-15). This was supported by single-cell analyses comparing early response to baricitinib (2 weeks) to baseline disease. Thus, shifts were observed in the myeloid and T-cell clusters (FIG. 7B, 7C). Keratinocytes, particularly in the basal layer of the epidermis, are the cellular target of T-cell responses in LP skin. 8 Basal layer keratinocytes (KRT5+) demonstrated two distinct states, “basal KC 1” and “basal KC 2” (FIG. 7D), with the “basal KC 1” state having an inflammatory state enriched for interferon and JAK1/JAK2 signaling and expression of MHC class I and class II molecules (FIG. 7F), which were absent in the “basal KC 2” state (FIG. 7G), suggesting that basal KC 1 keratinocytes are the main target of cytotoxic responses in LP. Strikingly, within only 2 weeks of baricitinib treatment there was a marked shift from “basal KC 1” state to “basal KC 2” state (FIG. 7E), likely reflecting suppression of IFN responses (FIG. 7F) and decreased antigen presentation (FIG. 7G).

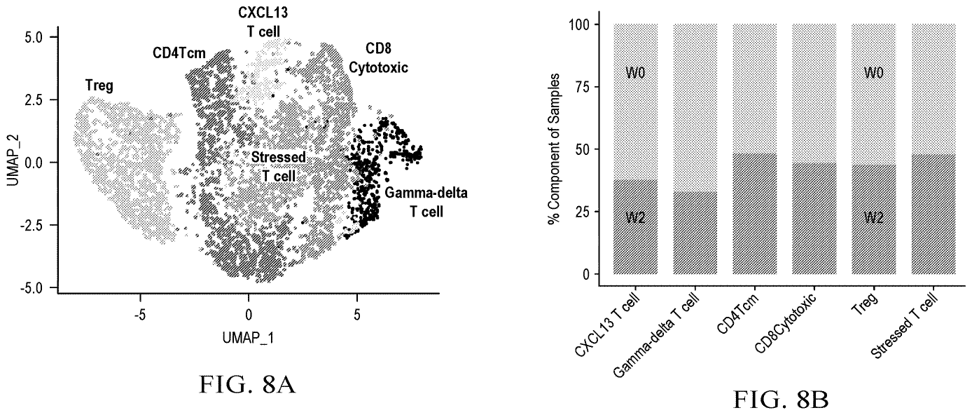

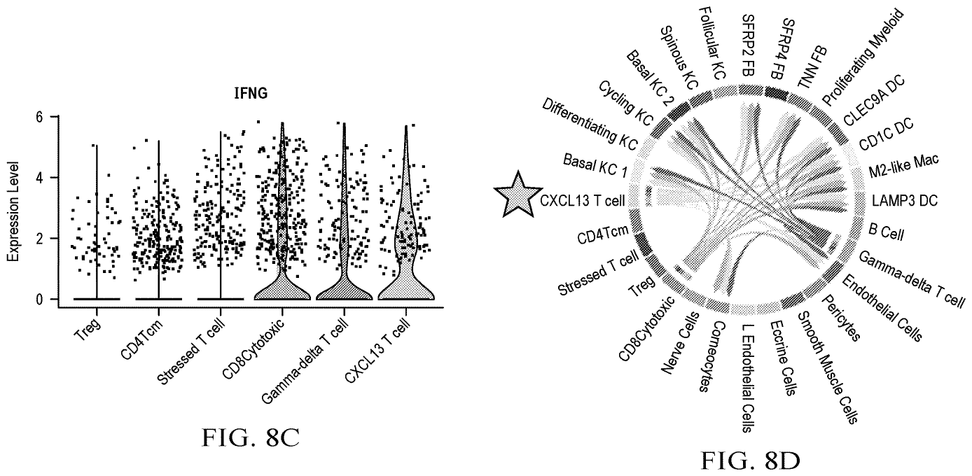

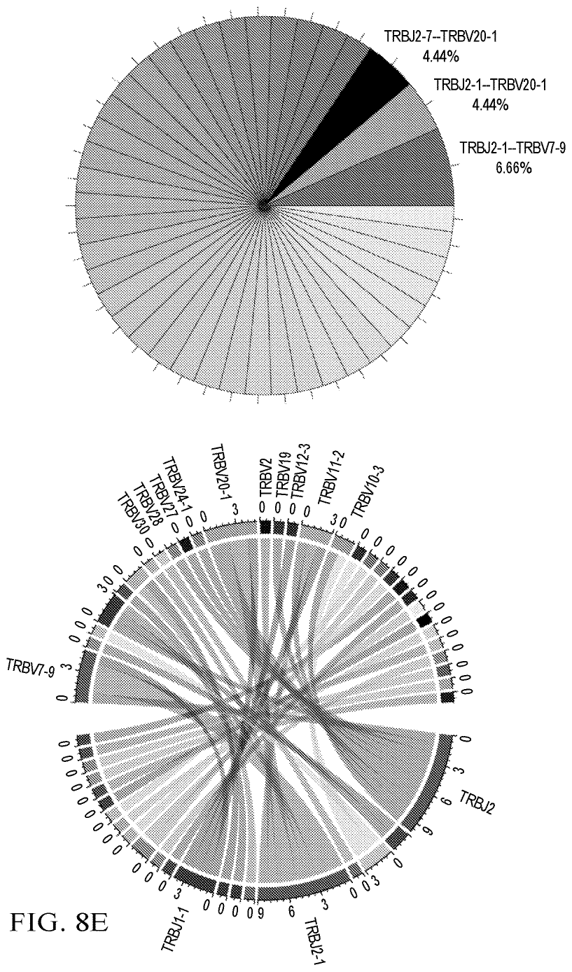

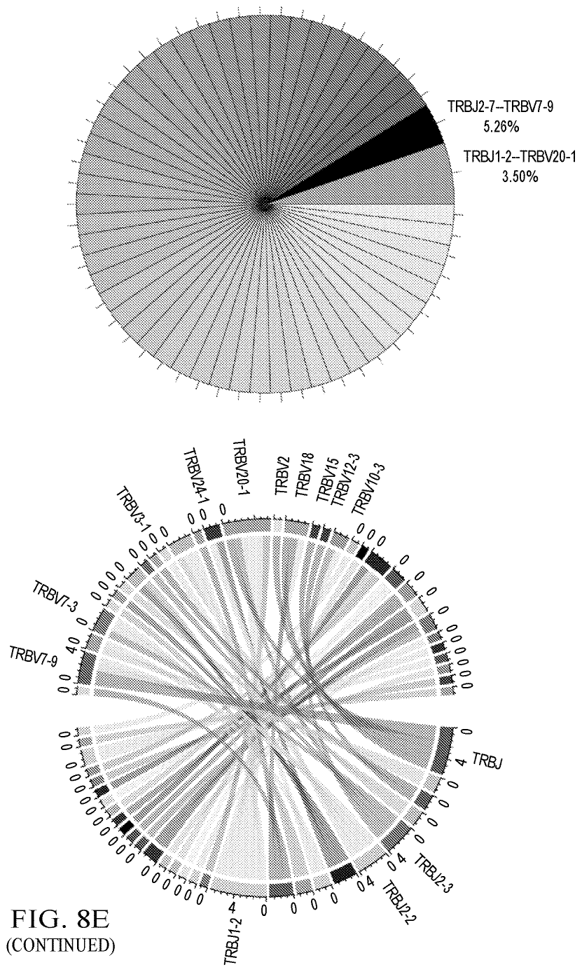

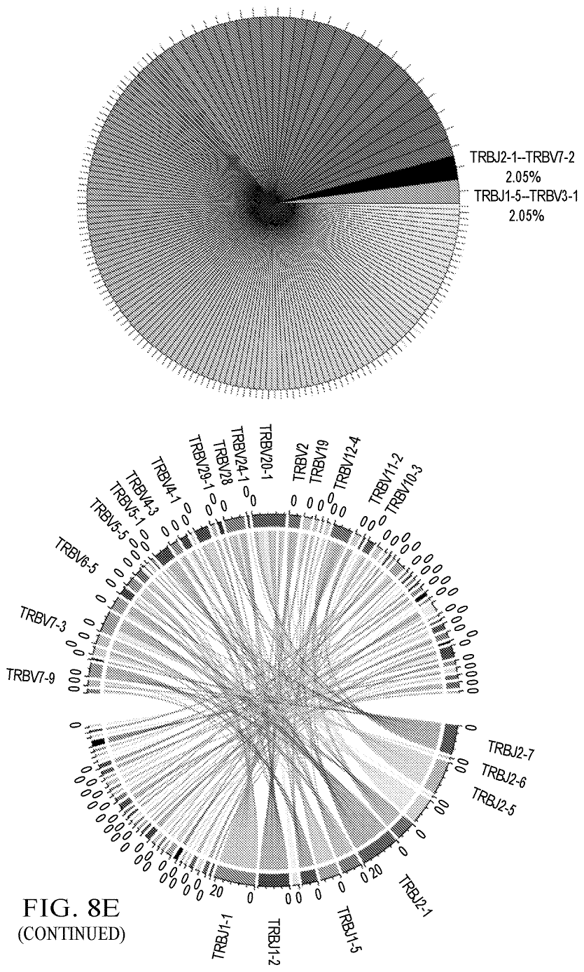

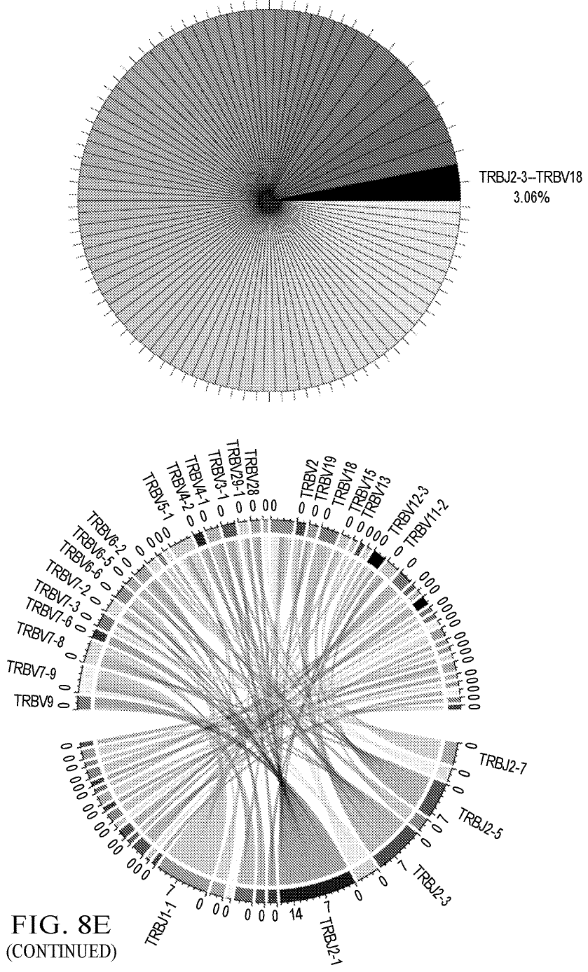

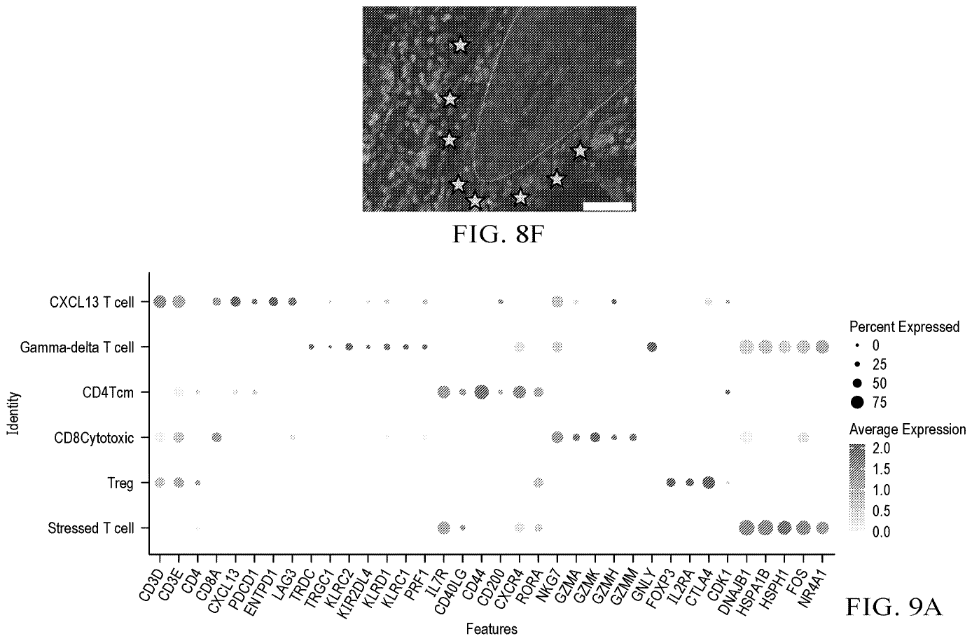

Prominent T-cell infiltration is a hallmark feature of LP histology, but the nature of the T-cell involvement has not previously been addressed. The inventors identified six subsets of T-cells in LP skin, including T regulator cells (T regs), CD4+ central memory T-cells, “stressed” T-cells, CD8 cytotoxic T-cells, gamma-delta T-cells, and a novel CXCL13+ T-cell subset (FIG. 8A, 8B). CD8 cytotoxic, gamma-delta, and CXCL13+ T-cell subsets were the main source of IFN-γ expression in LP skin (FIG. 8C) and demonstrated prominent cell-cell interactions with stromal cells, particularly basal keratinocytes (FIG. 8D). The T-cell subsets were localized at the dermal-epidermal junction with prominent expression of cytotoxic markers including GZMB, GZMA, and GNLY. Consistent with the T-cell response being directed against self-antigen(s), the CXCL13+CD8+ subset had evidence of oligoclonality in LP skin (FIG. 8E), and the proximity of CXCL13 T cells was confirmed to the epidermis in inflamed LP epidermis (FIG. 8F). The inventors did not observe prominent mRNA expression of other T-cell cytokines in LP, including the Th17 cytokines IL17A, IL17F, IL22, and IL26 or the Th2 cytokine IL4. Interestingly, IL13 mRNA expression was observed in central memory CD4+ T cells and the CXCL13 T cell subset, and in the bulk RNA-seq data (17.6-fold, FDR=3.3×10-08).

Five myeloid cell populations were identified in LP skin (M2-like, LAMP3, CD1C, CLEC9A, and proliferating myeloid cells) along with a small number of B-cells and three major fibroblast subsets (SFRP2, TNN, and SFRP4).

Consistent with LP being a systemic disease, a decrease in ISG expression was observed, including IFITM1, MHC class I (HLA-B), class II (HLA-DPA1), and the cytotoxic marker GNLY, with baricitinib treatment across multiple cell populations, along with decrease in biological processes including interferon signaling (p=1.5×10-8) in myeloid cells and MHC class II antigen presentation (p=9.4×10-4) in CD4+ T-cells, but no major shifts in cell populations was observed.

This example shows an open-label, single-arm trial demonstrated rapid and sustained response to baricitinib in cutaneous LP. Notably, the majority of the patients in the study had chronic, treatment-refractory LP, with half having failed systemic therapy, including methotrexate and oral or intramuscular corticosteroids. This rapid response included improvements in all measures of disease activity including physician global assessment, quality of life measures and pruritus. Only five patients required dose escalation of baricitinib to 4 mg daily. These data show a dose-dependent response to baricitinib in LP.

While the pathologic responses in LP are heavily skewed towards Th1 responses, IL-13, but not IL-4, was found to be elevated in LP skin, suggesting that it's immunopathogenesis is more complex than a simple Th1 polarization response, and shows that IL-13 may have a pro-pruritic role in LP, which is effectively ameliorated by baricitinib.

These data demonstrate that LP is an autoimmune disease with T cell responses directed against a pathogenic self-antigen. The primary source of IFN-γ in LP is cytotoxic CD8+ and gamma-delta T-cells. Here, a novel subset of CXCL13+CD8+ T-cells was identified that are a major source of IFN-γ in LP and show oligoclonality, showing reactivity against a limited set of possible autoantigens. Similar CXCL13+CD8+ T-cells have been described for tumor-reactive cells triggered by immune-checkpoint blockade. Interestingly, lichenoid dermatitis is not an uncommon cutaneous side-effect of immune-checkpoint inhibitor treatment, although the specific T-cell lymphocyte population in that setting has not been previously explored. Notably, the three CD8+ T-cell populations were localized at the dermal-epidermal junction and showed predicted interactions with basal layer keratinocytes where enriched IFN responses, and MHC class I and class II expression were observed. These findings show that IFN-γ signals priming basal cells towards cytotoxic attack, setting the stage for a vicious self-sustaining cycle of cytotoxic responses against basal keratinocytes.

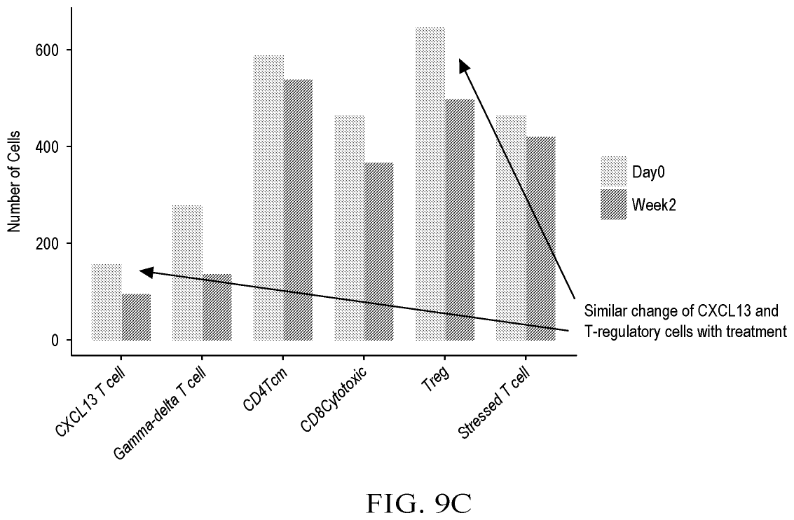

FIGS. 9A to 9C show regulatory T cells in a new pathogenic T-cell identified in LP. FIG. 9A shows Clean T-regulatory population by single cell sequencing. FIG. 9B shows the co-localization of CXCL13 and T-regulatory cells. FIG. 9C shows a similar change of CXCL13 and T-regulatory cells with treatment.

Here, the present inventors identified a novel subset of CXCL13+CD8+ T-cells that are a major source of IFN-γ in LP. It was found that JAK 1, 2 inhibitor disrupts the key interaction of the skin and inflammatory cells. Additional interactions are observed between skin and inflammatory cells and anti-inflammatory signaling. Further, the colocalization of T-regulatory cells and CXCL13+CD8+ T-cells is shown.

CXCL13+CD8+ T-cells have been described for tumor-reactive cells triggered by immune-checkpoint blockade in ⅓-½ of patients being treated for cancer. These cells may portend a poor prognosis in cancer and are associated with high tumor infiltrate of T-regulatory cells. These cells highly interact with T-regulatory cells in the tumor microenvironment and area associated with T-regulatory cell expansion. Regulate IL-10 secreting B-cells and play a key role in metastasis and treatment response.

The inventors did not observe evidence of involvement of IL-17 in LP pathogenesis, as previously suggested. However, most of the reports on IL-17 in LP have focused on oral LP, which was not included in this clinical trial.

Serious adverse events did not occur with baricitinib. A single AE of neutropenia was deemed probably related to the study drug, yet this was mild and did not result in treatment discontinuation.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EMBODIMENTS

Embodiment 1. A method, or a use of a composition, for treating cutaneous lichen planus in a mammal in need thereof, said method comprising orally administering, to said mammal, a composition comprising a janus kinase (JAK) inhibitor, wherein said administering results in a reduction in total lesion count from baseline.

Embodiment 2. The method of embodiment 1, wherein the mammal is a human.

Embodiment 3. The method of embodiment 2, wherein the human is an adult.

Embodiment 4. The method of any one of embodiments 1 to 3, wherein the JAK inhibitor is selected from the group consisting of baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, and povorcitinib.

Embodiment 5. The method of any one of embodiments 1 to 4, wherein the administering results in an improvement in the mammal's Index Treatment and Control Lesion by modified Clinical Assessment Scale of Severity for Index Lesion Signs and Symptoms (CAILS) score from baseline.

Embodiment 6. The method of any one of embodiments 1 to 5, wherein the mammal has clinical and histological features of lichen planus.

Embodiment 7. The method of any one of embodiments 1 to 6, wherein the mammal has a Body Surface Area of lichen planus involvement of up to 20% at baseline.

Embodiment 8. The method of any one of embodiments 1 to 6, wherein the mammal has a Body Surface Area of lichen planus involvement of 2% to 20% at baseline.

Embodiment 9. The method of any one of embodiments 1 to 8, wherein the mammal has a minimum of 4 lesions of lichen planus at baseline.

Embodiment 10. The method of any one of embodiments 1 to 8, wherein the mammal has a minimum of 10 lesions of lichen planus at baseline.

Embodiment 11. The method of any one of embodiments 1 to 10, wherein the mammal has had lichen planus for at least 8 months.

Embodiment 12. The method of any one of embodiments 1 to 11, wherein the mammal has failed to achieve an adequate response to prior therapy for lichen planus.

Embodiment 13. The method of embodiment 12, wherein the prior therapy is selected from the group consisting of one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide; topical tacrolimus and pimecrolimus; systemic immunosuppressant; oral metronidazole; oral sulfasalazine; and oral retinoid.

Embodiment 14. The method of any one of embodiments 1 to 13, wherein the administering results in a reduction in the mammal's total lesion count from baseline.

Embodiment 15. The method of any one of embodiments 1 to 14, wherein the administering is not topical.

Embodiment 16. The method of embodiment 3, wherein the baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, or povorcitinib is deuterated.

Embodiment 17. Use of a composition for treating cutaneous lichen planus in a mammal in need thereof, said method comprising orally administering, to said mammal, the composition comprising an amount of a Janus Kinase (JAK) inhibitor in a pharmacologically acceptable carrier sufficient to reduce a total lesion count from baseline.

Embodiment 18. The use of embodiment 17, wherein the mammal is a human.

Embodiment 19. The use of embodiment 18, wherein the human is an adult.

Embodiment 20. The use of any one of embodiments 17 to 19, wherein the JAK inhibitor is selected from the group consisting of baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, and povorcitinib.

Embodiment 21. The use of any one of embodiments 17 to 20, wherein the administering results in an improvement in the mammal's Index Treatment and Control Lesion by modified Clinical Assessment Scale of Severity for Index Lesion Signs and Symptoms (CAILS) score from baseline.

Embodiment 22. The use of any one of embodiments 17 to 21, wherein the mammal has clinical and histological features of lichen planus.

Embodiment 23. The use of any one of embodiments 17 to 22, wherein the mammal has a Body Surface Area of lichen planus involvement of up to 20% at baseline.

Embodiment 24. The use of any one of embodiments 17 to 22, wherein the mammal has a Body Surface Area of lichen planus involvement of 2% to 20% at baseline.

Embodiment 25. The use of any one of embodiments 17 to 24, wherein the mammal has a minimum of 4 lesions of lichen planus at baseline.

Embodiment 26. The use of any one of embodiments 17 to 24, wherein the mammal has a minimum of 10 lesions of lichen planus at baseline.

Embodiment 27. The use of any one of embodiments 17 to 26, wherein the mammal has had lichen planus for at least 8 months.

Embodiment 28. The use of any one of embodiments 17 to 27, wherein the mammal has failed to achieve an adequate response to prior therapy for lichen planus.

Embodiment 29. The use of embodiment 28, wherein the prior therapy is selected from the group consisting of one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide; topical tacrolimus and pimecrolimus; systemic immunosuppressant; oral metronidazole; oral sulfasalazine; and oral retinoid.

Embodiment 30. The use of any one of embodiments 17 to 13, wherein the administering results in a reduction in the mammal's total lesion count from baseline.

Embodiment 31. The use of any one of embodiments 17 to 14, wherein the administering is not topical.

Embodiment 32. The use of embodiment 20, wherein the baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, or povorcitinib is deuterated.

### REFERENCES

1. Mangold, A. R. and M. R. Pittelkow, Lichen planus. Clinical and Basic Immunodermatology, 2017: p. 551-576.
2. Boyd, A. S. and K. H. Neldner, Lichen planus. J Am Acad Dermatol, 1991. 25(4): p. 593-619.
3. Pietschke, K., et al., The inflammation in cutaneous lichen planus is dominated by IFN-γ and IL-21-A basis for therapeutic JAK1 inhibition. Exp Dermatol, 2021. 30(2): p. 262-270.
4. Groves, R. W., et al., Vascular cell adhesion molecule-1: expression in normal and diseased skin and regulation in vivo by interferon gamma. J Am Acad Dermatol, 1993. 29(1): p. 67-72.
5. Bennion, S. D., et al., In three types of interface dermatitis, different patterns of expression of intercellular adhesion molecule-1 (ICAM-1) indicate different triggers of disease. J Invest Dermatol, 1995. 105(1 Suppl): p. 71S-79S.
6. Shao, S., et al., IFN-γ enhances cell-mediated cytotoxicity against keratinocytes via JAK2/STAT1 in lichen planus. Sci Transl Med, 2019. 11(511).
7. Szabo, S. J., et al., Distinct effects of T-bet in TH1 lineage commitment and IFN-gamma production in CD4 and CD8 T cells. Science, 2002. 295(5553): p. 338-42.
8. Damsky, W., et al., Treatment of severe lichen planus with the JAK inhibitor tofacitinib. J Allergy Clin Immunol, 2020. 145(6): p. 1708-1710.e2.
9. Yang, C. C., et al., Tofacitinib for the treatment of lichen planopilaris: A case series. Dermatol Ther, 2018. 31(6): p. e12656.
10. Moussa, A., et al., Effective treatment of oral lichen planus with the JAK inhibitor baricitinib. Australas J Dermatol, 2022. 63(2): p. 276-277.
11. Balestri, R., et al., Treatment of Oral Erosive Lichen Planus With Upadacitinib. JAMA Dermatol, 2022. 158(4): p. 457-458.
12. Kooybaran, N. R., et al., Alleviation of erosive oral and esophageal lichen planus by the JAK1 inhibitor upadacitinib. J Dtsch Dermatol Ges, 2021. 19(12): p. 1778-1780.
13. Seiringer, P., et al., Tofacitinib in Hypertrophic Lichen Planus. Acta Derm Venereol, 2020. 100(14): p. adv00220.
14. Brumfiel, C. M., et al., Ruxolitinib Cream in the Treatment of Cutaneous Lichen Planus: A Prospective, Open-Label Study. J Invest Dermatol, 2022. 142(8): p. 2109-2116.e4.
15. Punchera, J. and E. Laffitte, Treatment of Severe Nail Lichen Planus With Baricitinib. JAMA Dermatol, 2022. 158(1): p. 107-108.
16. Moussa, A., et al., Treatment of lichen planopilaris with baricitinib: A retrospective study. J Am Acad Dermatol, 2022. 87(3): p. 663-666.
17. Olsen, E. A., et al., Clinical end points and response criteria in mycosis fungoides and Sézary syndrome: a consensus statement of the International Society for Cutaneous Lymphomas, the United States Cutaneous Lymphoma Consortium, and the Cutaneous Lymphoma Task Force of the European Organisation for Research and Treatment of Cancer. J Clin Oncol, 2011. 29(18): p. 2598-607.
18. Tsoi, L. C., et al., Atopic Dermatitis Is an IL-13-Dominant Disease with Greater Molecular Heterogeneity Compared to Psoriasis. J Invest Dermatol, 2019. 139(7): p. 1480-1489.
19. Ma, F., et al., Single cell and spatial sequencing define processes by which keratinocytes and fibroblasts amplify inflammatory responses in psoriasis. Nat Commun, 2023. 14(1): p. 3455.
20. Butler, A., et al., Integrating single-cell transcriptomic data across different conditions, technologies, and species. Nat Biotechnol, 2018. 36(5): p. 411-420.
21. Simpson, E. L., et al., Two Phase 3 Trials of Dupilumab versus Placebo in Atopic Dermatitis. N Engl J Med, 2016. 375(24): p. 2335-2348.
22. Chren, M. M., The Skindex instruments to measure the effects of skin disease on quality of life. Dermatol Clin, 2012. 30(2): p. 231-6, xiii.
23. Yosipovitch, G., et al., Peak Pruritus Numerical Rating Scale: psychometric validation and responder definition for assessing itch in moderate-to-severe atopic dermatitis. Br J Dermatol, 2019. 181(4): p. 761-769.
24. Soumelis, V., et al., Human epithelial cells trigger dendritic cell mediated allergic inflammation by producing TSLP. Nat Immunol, 2002. 3(7): p. 673-80.
25. Storan, E. R., et al., Role of cytokines and chemokines in itch. Handb Exp Pharmacol, 2015. 226: p. 163-76.
26. Oetjen, L. K., et al., Sensory Neurons Co-opt Classical Immune Signaling Pathways to Mediate Chronic Itch. Cell, 2017. 171(1): p. 217-228.e13.
27. Liu, B., et al., Single-cell meta-analyses reveal responses of tumor-reactive CXCL13 (+) T cells to immune-checkpoint blockade. Nat Cancer, 2022. 3(9): p. 1123-1136.
28. Schaberg, K. B., et al., Immunohistochemical analysis of lichenoid reactions in patients treated with anti-PD-L1 and anti-PD-1 therapy. J Cutan Pathol, 2016. 43(4): p. 339-46.
29. Husein-ElAhmed, H. and M. Steinhoff, Potential role of INTERLEUKIN-17 in the pathogenesis of oral lichen planus: a systematic review with META-analysis. J Eur Acad Dermatol Venereol, 2022. 36(10): p. 1735-1744.

1. A method for treating cutaneous lichen planus in a mammal in need thereof, said method comprising orally administering, to said mammal, a janus kinase (JAK) inhibitor, wherein said administering results in a reduction in total lesion count from baseline.

2. The method of claim 1, wherein the mammal is a human, or is a human adult.

3. (canceled)

4. The method of claim 1, wherein the JAK inhibitor is selected from the group consisting of baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, and povorcitinib.

5. The method of claim 1, wherein the administering results in an improvement in the mammal's Index Treatment and Control Lesion by modified Clinical Assessment Scale of Severity for Index Lesion Signs and Symptoms (CAILS) score from baseline.

6. The method of claim 1, wherein the mammal has at least one of: clinical and histological features of lichen planus, a Body Surface Area of lichen planus involvement of up to 20% at baseline, a Body Surface Area of lichen planus involvement of 2% to 20% at baseline, has a minimum of 4 lesions of lichen planus at baseline, has a minimum of 10 lesions of lichen planus at baseline, or has had lichen planus for at least 8 months.

7. (canceled)

8. (canceled)

9. (canceled)

10. (canceled)

11. (canceled)

12. The method of claim 1, wherein the mammal has failed to achieve an adequate response to prior therapy for lichen planus.

13. The method of claim 12, wherein the prior therapy is selected from the group consisting of one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide; topical tacrolimus and pimecrolimus; systemic immunosuppressant; oral metronidazole; oral sulfasalazine; and oral retinoid.

14. The method of claim 1, wherein the administering results in a reduction in the mammal's total lesion count from baseline.

15. The method of claim 1, wherein the administering is not topical.

16. The method of claim 2, wherein the baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, or povorcitinib is deuterated.

17. A method of treating cutaneous lichen planus in a mammal in need thereof, said method comprising orally administering, to said mammal, a composition comprising an amount of a Janus Kinase (JAK) inhibitor in a pharmacologically acceptable carrier sufficient to reduce a total lesion count from baseline.

18. The method of claim 17, wherein the mammal is a human, or is a human adult.

19. (canceled)

20. The method of claim 17, wherein the JAK inhibitor is selected from the group consisting of baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, and povorcitinib.

21. The method of claim 17, wherein the administering results in an improvement in the mammal's Index Treatment and Control Lesion by modified Clinical Assessment Scale of Severity for Index Lesion Signs and Symptoms (CAILS) score from baseline.

22. The method of claim 17, wherein the mammal has at least one of: clinical and histological features of lichen planus, a Body Surface Area of lichen planus involvement of up to 20% at baseline, a Body Surface Area of lichen planus involvement of 2% to 20% at baseline, has a minimum of 4 lesions of lichen planus at baseline, has a minimum of 10 lesions of lichen planus at baseline, or has had lichen planus for at least 8 months.

23. (canceled)

24. (canceled)

25. (canceled)

26. (canceled)

27. (canceled)

28. The method of claim 17, wherein the mammal has failed to achieve an adequate response to prior therapy for lichen planus.

29. The method of claim 28, wherein the prior therapy is selected from the group consisting of one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide; topical tacrolimus and pimecrolimus; systemic immunosuppressant; oral metronidazole; oral sulfasalazine; and oral retinoid.

30. The method of claim 17, wherein the administering results in a reduction in the mammal's total lesion count from baseline.

31. The method of claim 17, wherein the administering is not topical.

32. The method of claim 20, wherein the baricitinib, ruxolitinib, tofacitinib, upadacitinib, fedratinib, pacritinib, momelotinib, abrocitinib, delgocitinib, filgotinib, peficitinib, deucravacitinib, brepocitinib, SHR0302, itacitinib, or povorcitinib is deuterated.
